(19) United States
(12) Patent Application Publication
(10) Pub. No.: US 2006/0281820 A1
(43) Pub. Date: Dec. 14, 2006

(54) Lfa-1 inhibitors and use thereof

(76) Inventor: Kuniyasu Soda, Saitama (JP)

Correspondence Address: STEPTOE & JOHNSON LLP, 1330 CONNECTICUT AVENUE, N.W., WASHINGTON, DC, 20036, US

(21) Appl. No.: 10/545,024

(22) PCT Filed: Feb. 19, 2003
(86) PCT No.: PCT/JP03/01780

§ 371 Date: May 23, 2006

(51) Int. Cl. A61K 31/13

(52) U.S. Cl. 514/674

(57) ABSTRACT

The present invention provides an LFA-1 inhibitor comprising at least one member selected from the group consisting of polyamines represented by general formula (1) and pharmaceutical acceptable salts thereof: NH₂—(CH₂)m1-(NH)p1-(CH₂)m2-(NH)p2-(CH₂)m3-(NH)p3-(CH₂)m4-(NH)p4-(CH₂)m5-NH₂ (1), wherein at least two of m1 to m5 exceed 0, each of m1 to m5 is independently an integer of 0 to 7, the sum of m1+m2+m3+m4+m5 is 2 or more but less than 18, at least one of p1, p2, p3 and p4 is 1, and each of others independently represents 0 or 1. The present invention also provides a pharmaceutical composition containing the LFA-1 inhibitor and a method for the inhibition or the treatment of diseases.

### TECHNICAL FIELD

### Field of the Invention

The present invention relates to LFA-1 inhibitors and use thereof. In particular, the present invention relates to a selective function inhibitor of LFA-1 containing polyamines, a pharmaceutical composition containing the LFA-1 inhibitor and a method for the inhibition or the treatment of diseases comprising the use of the LFA-1 inhibitor and the pharmaceutical composition.

### BACKGROUND ART

### Background of the Invention

In general, a cell membrane differentiation antigen (Cluster of differentiation: called CD hereinafter), which plays an important role in function and differentiation of the cells, is expressed in a surface of cells. The CD includes adhesion molecules necessary as extracellular matrix for cell-cell adhesion. It has been clarified that the adhesion molecules not only contribute to cell-cell adhesion but also have important functions of precisely regulating various reactions of the body such as development and immune response by their action on intracellular signal transduction systems.

Heretofore, a number of adhesion molecules have been identified, of which LFA-1 composed of cell membrane differentiation antigens CD11a and CD18 has been shown to serve an important function in formation of inflammation. It has been clarified that LFA-1 particularly existing in immunocytes such as peripheral blood monocyte (lymphocyte, monocyte, and macrophage) has a central role in a process from the very early stage to progression of various inflammations.

As described below, it has been clarified that these LFA-1 molecules are factors, which play a central role in formation and progression of many diseases such as arteriosclerosis, rejection of graft in organ transplantation, autoimmune disease, and allergic disease (1: reference number, it is described in the back of this specification as reference).

The LFA-1 is highly expressed in cells responsible for immunity, such as lymphocyte, monocyte, macrophage, and granular leukocyte, and there usually exists an adhesion partner to be paired with the LFA-1, that is, another adhesion molecule selectively binding to the LFA-1 adhesion molecule. The signal transduction into cells starts by adhesion of this pair of adhesion molecules, and the cells are activated. It has been clarified that the LFA-1 binds selectively to the adhesion molecule called CD54 (another name ICAM-1: Intercelullar adhesion molecule-1) highly expressed in vascular endothelial cell etc (2). In the case, ICAM-1 is called ligand of LFA-1.

In the early stage of inflammation, immunocytes such as lymphocyte, monocyte, macrophage, and granular leukocyte (granulocyte) having LFA-1 on the surface are activated by adhesion to vascular endothelial cell and the like expressing the ligand ICAM-1, and the inflammation is caused in the tissues. When the inflammation in the tissues starts by this reaction, activation of other adhesion molecules and functional cell membrane differentiation antigen molecules is induced by production of mediator inducing various inflammations, and the inflammations are increased. Therefore, development and progression of the inflammation can be inhibited by decreasing expression levels of LFA-1 or by binding antibodies and functional molecules to this adhesion molecule and decreasing the adhesive function of the adhesion molecule (3, 4, 5). In contrast, the inflammation can also be increased by increasing the expression of the LFA-1 (6).

It has further been found that LFA-1 has an important role in antitumor activity exhibited by immunocytes. It has been well known that the immunocytes recognize and kill cancer cells by culturing the immunocytes together with the cancer cells in the same cell culture medium. When the function of LFA-1 is inhibited by adding antibodies to LFA-1 to this culture medium, a part of antitumor activity to the cancer cells is decreased (7).

Thus, the discovery of means for increasing the expression of LFA-1 can pave the way for establishment of a method for the treatment of cancer and a therapy by which antimicrobial activity is increased by causing a high level of inflammation. As a consequence, cell membrane differentiation antigen molecules (cluster of differentiation antigenic molecules), particularly LFA-1, in immunocytes have been targets of great importance to the treatment of inflammatory disease and cancer.

As described above, LFA-1 is a complex of CD11a and CD18. While the involvement of LFA-1 in a variety of diseases described above has been revealed by documents listed below, it has been clarified that the function of LFA-1 can be inhibited by inhibiting the expression of CD11a and CD18 composing the LFA-1, particularly the expression of CD11a.

As described above, because the deep involvement of LFA-1 in development and progression of inflammation has been clarified, a number of attempts have been made so far on development of antibodies and functional molecules, which decrease the function of LFA-1, for the purpose of preventing and treating various inflammatory diseases.

In fact, therapeutic agents have been developed actively in order to achieve the inhibition of onset and the treatment of diseases for which LFA-1 has already been found to have a central role in development and progression of the pathology (arteriosclerosis, rejection of graft, autoimmune disease (type I diabetes (insulin-dependent diabetes mellitus), thyroid disease, autoimmune arthritis, cerebrospinal peripheral neuritis or degenerative disease), and the like), the treatment and inhibition of allergic disease, the inhibition of ischemic reperfusion injury, the inhibition and reduction of progression of hypertensive nephropathy and diabetic retinopathy, etc, by inhibiting LFA-1 or CD11a composing the LFA-1.

As described below, the inhibition of progression of arteriosclerosis, the inhibition of rejection of graft, and the improvement of symptoms of one of autoimmune diseases (psoriasis: a kind of dermatitis), etc, have already been achieved by administering anti-LFA-1 antibodies, function inhibitors of LFA-1, or synthesized small molecules to humans. For each disease or pathology of autoimmune disease (type I diabetes, Graves' disease (Basedow disease), Hashimoto disease, autoimmune arthritis, cerebrospinal peripheral neurodegenerative disease and the like), allergic disease, ischemic reperfusion injury, and diabetic retinopathy, it has further been confirmed that administration of anti-LFA-1 antibodies or substances having anti-LFA-1 effects to animals having the same pathology as these human diseases makes the inhibition of the disease or the improvement of the symptoms possible.

As described above, it has been approved that a method for the inhibition of the adhesion of LFA-1 to its ligand ICAM-1 by using the antibodies and the small molecules is effective for the treatment of many diseases. Development of the methods for the inhibition of function of LFA-1 and the therapeutic agents has been advanced rapidly and extensively. In fact, the antibodies to LFA-1 have been developed and have already been started to use for the treatment of human diseases (8, 9, 10, 11, 12).

Similarly, a material having a structure similar to the structure of ICAM-1 serving as ligand of LFA-1 has been synthesized, and the materials such as inhibiting LFA-1 adhesion to ICAM-1 by fitting in a keyhole of LFA-1 and not inducing activation of cell function via LFA-1 have also been purified (13).

It has been clarified that an agent for the treatment of hyperlipemia (metabolic disorder increasing neutral fat and cholesterol in blood and inducing arteriosclerosis such as cardiac infarction) inhibits the function of LFA-1. It has been reported that progression of arteriosclerosis of a patient internally taking this agent is inhibited, and graft survival rate of organ transplantation is enhanced (14, 15, 16).

However, it has been found that function inhibition and cell adhesion inhibition of LFA-1 by the agent for the treatment of hyperlipemia (generally, called statin drug) are only exerted in the high concentration, which is not able to exist in a human body. The antibodies and the small molecules are materials not essentially existing in the natural world and are foreign bodies in the human body, and it has been pointed out that when they are used in the body, it is unknown what side effect appears (17).

In the history of medical care, it is a well known fact that serious side effects attributed to such substances have previously endangered many lives. Accordingly, the application of these substances to humans requires studies on safety including extensive clinical tests.

Furthermore, all of these substances adhere directly to LFA-1 molecules from the outside of the cells and physically delete the adhesive function. However, the function of LFA-1 is regulated through stimulation to the cell having this adhesion molecule, which triggers the transmission from the inside of the cell of information for increasing expression of this LFA-1 molecule. Namely, a substance named chemokine secreted from immunocytes as required by the living body reacts with chemokine receptor existing on the surface of the cell having LFA-1, and a signal of LFA-1 activation is thereby sent from the inside of the cell. As a result, LFA-1 is activated, and the adhesive function of the cell is enhanced. Accordingly, treatment with an agent forcedly putting LFA-1 in a mold from the outside and allowing the LFA-1 to fall into an immobilized state completely blocks physiological reaction in the living body. For putting the treatment into practical use, dangerous side effects might be caused unless extensive safety for humans is confirmed.

For example, when mice not expressing LFA-1 in the cells are produced by gene manipulation, transfer of the tumor cell transplanted in this mouse is facilitated more than that of normal mice (18).

It has already been reported that when the antibodies to LFA-1 are administered to infected animals, the symptoms are exacerbated, and resistance to bacterial infection is decreased. It has been pointed out that there is a possibility to cause serious problems for LFA-1 forcedly non-functionalized (19).

Under these circumstances, there was a need for development of an inhibitor, which inhibits the expression or function of LFA-1 with safety and effect.

### DISCLOSURE OF THE INVENTION

The present inventor has conducted studies in consideration of said circumstances and has consequently gained findings that a polyamine existing in the natural world and being ingested together with foods by humans inhibits the expression of cell membrane differentiation antigens CD11a and CD18 and as a result, inhibits the function of an LFA-1 adhesion molecule. The present invention has been completed based on the findings.

An object of the present invention is to provide an LFA-1 inhibitor comprising a polyamine, a pharmaceutical composition comprising the LFA-1 inhibitor, and a method for the inhibition and the treatment of diseases with the LFA-1 inhibitors and the pharmaceutical compositions.

A further object of the present invention is to provide a selective function inhibitor of LFA-1 containing polyamines, a pharmaceutical composition for the treatment of arteriosclerosis comprising the LFA-1 inhibitors, a pharmaceutical composition for the inhibition of rejection comprising the LFA-1 inhibitors, a pharmaceutical composition for the treatment of autoimmune disease comprising the LFA-1 inhibitors, a pharmaceutical composition for the treatment of allergy comprising the LFA-1 inhibitors, a pharmaceutical composition for the treatment of ischemic reperfusion injury comprising the LFA-1 inhibitors, and a pharmaceutical composition for the treatment of diabetic retinopathy comprising the LFA-1 inhibitors.

It is a still further object of the present invention to provide a method for the inhibition and the treatment of diseases selected from the group consisting of arteriosclerosis, autoimmune disease, allergy, ischemic reperfusion injury, and diabetic retinopathy comprising administrating said LFA-1 inhibitors as well as a method for the inhibition of rejection comprising administrating said LFA-1 inhibitors.

In order to achieve said objects, the present invention provides the following invention.

The present invention provides an LFA-1 inhibitor comprising at least one member selected from the group consisting of polyamines having 2 to 6 amino groups and one or more linear or branched alkylene moieties having 2 to 7 carbon atoms and pharmaceutical acceptable salts thereof.

The present invention provides an LFA-1 inhibitor comprising at least one member selected from the group consisting of polyamines represented by the following general formula (1) and pharmaceutical acceptable salts thereof:
NH₂—(CH₂)m1-(NH)p1(CH₂)m2-(NH)p2-(CH₂)m3-(NH)p3-(CH₂)m4-(NH)p4-(CH₂)m5-NH₂ (1)
wherein each of m1 to m5 is independently an integer of 0 to 7, at least two of m1 to m5 exceed 0, the sum of m1+m2+m3+m4+m5 is 2 or more but less than 18, at least one of p1, p2, p3 and p4 is 1, and each of others independently represents 0 or 1.

The present invention further provides said LFA-1 inhibitor wherein the polyamine is selected from the group consisting of 3,3′-iminobispropylamine, N-aminobutyl-1,3-diaminopropane, 4,4′-iminobisbutylamine and N-aminopentyl-1,3-diaminopropane.

The present invention further provides said LFA-1 inhibitor wherein the polyamine is selected from the group consisting of 4,9-diazatridecane-1,13-diamine, 4,9-diazadodecane-1,12-diamine, 4,8-diazadodecane-1,12-diamine, 5,9-diazatridecane-1,13-diamine, 4,9-diazatridecane-1,13-diamine, 4,10-diazatridecane-1,13-diamine, 4,9-diazatridecane-1,13-diamine, 5,9-diazatridecane-1,13-diamine and 5,9-diazatridecane-1,14-diamine.

The present invention further provides said LFA-1 inhibitor wherein the polyamine is selected from the group consisting of 4,8,12-triazapentadecane-1,15-diamine, 4,8,12-triazahexadecane-1,16-diamine, 4,9,13-triazaheptadecane-1,17-diamine, 4,9,14-triazaoctadecane-1,18-diamine, 5,9,13-triazaheptadecane-1,17-diamine, 5,9,14-triazaoctadecane-1,18-diamine, 4,9,14-triazaoctadecane-1,18-diamine, and 5,10,14-triazaoctadecane-1,18-diamine.

The present invention further provides said LFA-1 inhibitor wherein the polyamine is selected from the group consisting of 4,8,12,16-tetraazanonadecane-1,19-diamine, 4,8,12,16-tetraazaicosane-1,20-diamine, 4,8,12,17-tetraazaicosane-1,20-diamine and 4,8,12,17-tetraazaicosane-1,20-diamine.

The present invention further provides a pharmaceutical composition for the treatment of arteriosclerosis, a pharmaceutical composition for the inhibition of rejection, a pharmaceutical composition for the treatment of autoimmune disease, a pharmaceutical composition for the treatment of allergy, a pharmaceutical composition for the treatment of ischemic reperfusion injury, and a pharmaceutical composition for the treatment of diabetic retinopathy comprising said LFA-1 inhibitor.

The present invention further provides a method for the treatment of diseases selected from the group consisting of arteriosclerosis, autoimmune disease, allergy, ischemic reperfusion injury, and diabetic retinopathy comprising administrating said LFA-1 inhibitor.

The present invention further provides a method for the inhibition of diseases selected from the group consisting of arteriosclerosis, autoimmune disease, allergy, ischemic reperfusion injury, and diabetic retinopathy comprising administrating said LFA-1 inhibitor.

The present invention further provides a method for the inhibition of rejection characterized by comprising administrating said LFA-1 inhibitor. definition

The terms used herein are defined as follows.

The term “polyamine” means a compound containing three or more amino groups and two or more linear or branched alkylene moieties having 2 to 7 carbon atoms in the same molecule.

The term “pharmaceutical acceptable salts” means nontoxic acid addition salts of mineral acid or organic acid that can be used pharmaceutically.

The term “patient” means warm-blooded animals such as mammals of interest of treatment. Examples of animals that fall within the scope of the patient include dogs, cats, rats, house mice, horses, cattle, sheep, and humans.

The term “CD11a” is called LFA-1α-chain, gp180/95, αL Integrin or the like as another name, and these names are unified into the name CD11a in the present specification and claims.

The term “CD18” is called LFA-1 β-chain, Integrin β2 or the like as another name, and these names are unified into the name CD18 in the present specification and claims.

### BRIEF DESCRIPTION OF THE DRAWINGS

FIG. 1 shows change (decrease) of average fluorescence intensity (measurement value) of CD11a of human peripheral blood monocyte cultured in cell culture medium with added spermine for 70-80 hours;

FIG. 2 shows change of CD11a histogram depending on spermine. It is shown that the number of the cells strongly expressing CD11a is decreased;

FIG. 3 shows change (decrease) of average fluorescence intensity of the cells expressing CD18 in the presence of spermine;

FIG. 4 shows change (decrease) of average fluorescence intensity (measurement value) of CD11a of human peripheral blood monocyte cultured in cell culture medium with added spermine, spermidine or putrescine for 70-80 hours;

FIG. 5 shows that average fluorescence intensity of CD11a of peripheral blood monocyte when cultured with spermine for 20-26 hours has no change;

FIG. 6 shows change (decrease) of average fluorescence intensity of CD11a of human peripheral blood monocytes which after cultured for 16-24 hours, were washed to remove spermine present outside of the cells, followed by culture in culture medium without spermine for 48-56 hours;

FIG. 7 shows change of average fluorescence intensity of the cells expressing adhesion molecules by spermine;

FIG. 8 shows change of average fluorescence intensity of functional cell membrane differentiation antigen except adhesion molecules by spermine;

FIG. 9 shows expression rate of CD11a positive cell and average fluorescence intensity of CD11a immediately after collection of blood and after culture for 72 hours;

FIG. 10 shows the rate (expression positive rate) to the total cells of cells that expressed adhesion molecules. The expression rate of adhesion molecules of the cells cultured with spermine was not decreased;

FIG. 11 shows the rate (expression positive rate) to the total cells of the cells that expressed functional cell membrane differentiation antigen except adhesion molecules. The expression rate of cell membrane differentiation antigen of the cells cultured with spermine was not decreased;

FIG. 12 shows change of average fluorescence intensity depending on spermine in the experiment using peripheral blood monocyte accidentally infected during culture or in the body before collection of blood. As shown in FIGS. 7 and 8, the expression of the cell membrane differentiation antigen such as CD16, 31, 49d, and 54, which is not usually decreased, is markedly decreased in the cells cultured with spermine;

FIG. 13 shows change of adhesive rate to culture plate of peripheral blood monocyte in the presence of spermine, spermidine, or putrescine;

FIG. 14 shows a result of increasing, by centrifugation of culture plate, inhibition effect on cell adhesion to the culture plate by spermine;

FIG. 15 shows that inhibition effect on adhesion to culture plate of the cells cultured with spermine for 20-24 hours is not apparent;

FIG. 16 shows change of adhesive (rate) to vascular endothelial cells of peripheral blood monocytes cultured with spermine for 20 hours or 72 hours. The inhibition effect on adhesion of the cells is not recognized in the culture for approximately 20 hours;

FIG. 17 shows change of the number of adhesive cells (actual number) to vascular endothelial cells of human peripheral blood monocytes which after cultured with spermine for 16-24 hours, were washed to remove spermine present outside of the cells, followed by culture in culture medium without spermine for 48-56 hours;

FIG. 18 shows change of adhesive (rate) to vascular endothelial cells of peripheral blood monocytes cultured with spermine, spermidine, or putrescine for 70-80 hours;

FIG. 19 shows change of cellular cytotoxicity and blastogenesis depending on spermine; and

FIG. 20 shows change of polyamine concentration in peripheral blood monocyte cultured with polyamines (spermine, spermidine, or putrescine).

### BEST MODE FOR CARRYING OUT THE INVENTION

Polyamine

Compounds useful to LFA-1 inhibitors, pharmaceutical compositions, and methods of the present invention are known in the field of chemistry, and most of them are available commercially. Compounds used in the present invention are, for the most part, biogenic amines which generally exist in organisms and can be produced by extraction from organisms, and the process is disclosed in literature such as Beilsteins Handbuch Der Organischen Chemie. Also, information of compounds used in the present invention is disclosed in Merck Index the ninth edition and Method in Molecular Biology (Vol. 79, Polyamine Protocols, Edited by: D. Morgan, Humana Press Inc., Totowa, N.J.), etc. It is apparent to those skilled in the art that polyamines of the present invention can be produced easily based on their information.

Polyamines used in the present invention are compounds having 3 to 6 amino groups and two or more linear or branched alkylene moieties having 2 to 7 carbon atoms. The polyamines include compounds having the following chemical formula (1):
NH₂—(CH₂)m1-(NH)p1-(CH₂)m2-(NH)p2-(CH₂)m3-(NH)p3-(CH₂)m4-(NH)p4-(CH₂)m5-NH₂ (1)
wherein at least two of m1 to m5 exceed 0, each of m1 to m5 is independently an integer of 0 to 7, preferably an integer of 0 to 5, the sum of m1+m2+m3+m4+m5 is 2 or more but less than 18, preferably 2 or more but less than 17, more preferably 4 or more but less than 16, at least one of p1, p2, p3 and p4 is 1, and each of others independently represents 0 or 1.

Polyamines of the present invention include a compound wherein m1 and m2 are an integer of 2 to 7, in particular an integer of 3 to 5, m3, m4 and m5 are 0, p1 is 1, and p2, p3 and p4 are 0 in the formula (1).

Polyamines of the present invention include a compound wherein m1, m2 and m3 are an integer of 2 to 7, in particular an integer of 3 to 5, m4 and m5 are 0, p1 and p2 are 1, and p3 and p4 are 0 in the formula (1).

Polyamines of the present invention include a compound wherein m1, m2, m3 and m4 are an integer of 2 to 7, in particular an integer of 3 to 5, m5 is 0, p1 to p3 are 1, and p4 is 0 in the formula (1).

The polyamines of the present invention include triamine, tetraamine, pentaamine and hexamine, and can be used alone or in combination. The polyamines of the present invention are specifically described below. References in patenthesis [ ] following each of the compounds are references describing a process for the production therefor.

Examples of the triamines used in the present invention include the followings:

Calgene (Norspermidine)

[Biochem Biophys. Res. Commun. 63. 69(1975)]

NH₂(CH₂)₃NH(CH₂)₃NH₂

(3,3′-iminobispropylamine)

Spermidine [Beil. 4 (2) 704]

NH₂(CH₂)₃NH(CH₂)₄NH₂

(N-aminobutyl-1,3-diaminopropane)

Homospermidine

NH₂(CH₂)₄NH(CH₂)₄NH₂

(4,4′-iminobisbutylamine)

Aminopropylcadaverine

NH₂(CH₂)₃NH(CH₂)₅NH₂

N-aminopentyl-1,3-diaminopropane

The most preferable triamine is spermidine among the above.

Examples of tetraamines used in the present invention include the following:

Thermine (norspermine)

NH₂(CH₂)₃NH(CH₂)₃NH(CH₂)₃NH₂

4,8-diazaundecane-1,11-diamine

Spermine [Beil. 4 (2) 704, Merck Index, 9.8515]

NH₂(CH₂)₃NH(CH₂)₄NH(CH₂)₃NH₂

4,9-diazadodecane-1,12-diamine

Thermospermine

NH₂(CH₂)₃NH(CH₂)₃NH(CH₂)₄NH₂

4,8-diazadodecane-1,12-diamine

Canavalmine

NH₂(CH₂)₄NH(CH₂)₃NH(CH₂)₄NH₂

5,9-diazatridecane-1,13-diamine

Aminopentylnorspermidine

NH₂(CH₂)₃NH(CH₂)₃NH(CH₂)₅NH₂

4,8-diazatridecane-1,11-diamine

N,N′-bis(aminopropyl)cadaverine

NH₂(CH₂)₃NH(CH₂)₅NH(CH₂)₃NH₂

4,10-diazaundecane-1,13-diamine

Aminopropylhomospermine

NH₂(CH₂)₃ NH(CH₂)₄NH(CH₂)₄NH₂

4,9-diazatridecan-1,13-diamine

Canavalmine

NH₂(CH₂)₄ NH(CH₂)₃NH(CH₂)₄NH₂

5,9-diazatridecan-1,13-diamine

Homospermine

NH₂(CH₂)₄NH(CH₂)₄NH(CH₂)₄NH₂

5,10-diazatetradecan-1,14-diamine

The preferable tetraamines are Thermine, Spermine, Homospermine, Thermospermine, Aminopentylnorspermidine, and N,N′-bis(aminopropyl)cadaverine. The more preferably tetraamine is spermine.

Examples of pentaamines used in the present invention include the followings:

Caldopentamine

NH₂(CH₂)₃NH(CH₂)₃NH(CH₂)₃NH(CH₂)₃NH₂

4,8,12-triazapentadecane-1,15-diamine

Homocaldopentamine

NH₂(CH₂)₃NH(CH₂)₃NH(CH₂)₃NH(CH₂)₄NH₂

4,8,12-triazahexadecane-1,16-diamine

Aminopropylcanavalmine

NH₂(CH₂)₃NH(CH₂)₄NH(CH₂)₃NH(CH₂)₄NH₂

4,9,13-triazaheptadecane-1,17-diamine

Bis(aminopropyl)homospermidine

NH₂(CH₂)₃NH(CH₂)₄NH(CH₂)₄NH(CH₂)₄NH₂

4,9,14-triazaoctadecane-1,18-diamine

Bis(aminobutyl)norspermidine

NH₂(CH₂)₄NH(CH₂)₃NH(CH₂)₃NH(CH₂)₄NH₂

5,9,13-triazaheptadecane-1,17-diamine

Aminobutylcanavalmine

NH₂(CH₂)₄NH(CH₂)₃NH(CH₂)₄NH(CH₂)₄NH₂

5,9,14-triazaoctadecane-1,18-diamine

Aminopropylhomospermine

NH₂(CH₂)₃NH(CH₂)₄NH(CH₂)₄NH(CH₂)₄NH₂

4,9,14-triazaoctadecane-1,18-diamine

Homopentamine

NH₂(CH₂)₄NH(CH₂)₄NH(CH₂)₃NH(CH₂)₄NH₂

5,10,14-triazaoctadecane-1,18-diamine

The preferable pentaamines are caldopentamine and homocaldopentamine.

Examples of hexaamines used in the present invention include the followings:

Caldohexamine

NH₂(CH₂)₃NH(CH₂)₃NH(CH₂)₃NH(CH₂)₃NH(CH₂)₃NH₂

4,8,12,16-tetraazanonadecane-1.19-diamine

Homocaldohexamine

NH₂(CH₂)₃NH(CH₂)₃NH(CH₂)₃NH(CH₂)₃NH(CH₂)₄NH₂

4,8,12,16-tetraazaicosane-1.20-diamine

Thermohexamine

NH₂(CH₂)₃NH(CH₂)₃NH(CH₂)₃NH(CH₂)₄NH(CH₂)₃NH₂

4,8,12,17-tetraazaicosane-1.20-diamine

Homethermohexamine

NH₂(CH₂)₃NH(CH₂)₃NH(CH₂)₄NH(CH₂)₃NH(CH₂)₃NH₂

4,8,13,16-tetraazaicosane-1.20-diamine

The preferable hexaamines are caldohexamine and homocaldohexamine.

In the present invention, said polyamines may be used in the pharmaceutically acceptable salt. The salts may be addition salts of organic acid or mineral acid, and include, for example, mineral addition salts such as hydrochloric acid, hydrofluoric acid, sulfuric acid, nitric acid, phosphoric acid, and for example, organic addition salts such as sulfonic acid, methansulfonic acid, sulfamic acid, tartaric acid, fumaric acid, hydrobromic acid, glycolic acid, citric acid, maleic acid, phosphoric acid, succinic acid, acetic acid, benzoic acid, ascorbic acid, p-toluenesulfonic acid, benzenesulfonic acid, naphthalenesulfonic acid, propionic acid, lactic acid, pyruvic acid, oxalic acid, stearic acid, cinnamic acid, aspartic acid, salicylic acid, and gluconic acid.

The salts are advantageous in the treatment because those are fume-free of free base. In particular, hydrochloric acid addition salt is the most preferable. As well known in the field of the invention, the acid addition salts can be easily prepared by contact of polyamines of free base form with suitable acid.

(Dose)

A dose of the LFA-1 inhibitor of the present invention is changed appropriately according to route of administration, and sex, symptoms, age and body weight of the patient. Generally, the dose is 0.01-100 mg/Kg of body weight, in particular 0.05-40 mg/kg of body weight, and more preferably 0.05-4 mg/kg of body weight as the polyamines, per day for a human adult. In the LFA-1 inhibitors and the pharmaceutical compositions of the present invention, said polyamines or combinations thereof as active ingredients can be used alone or in combination with other desired agents.

Preparation Form

The LFA-1 inhibitors or the pharmaceutical compositions of the present invention can be administered orally or parenterally. The form of the parenteral administration includes administration by injection such as instillation, intravenous injection, hypodermic injection, and intramuscular injection, percutaneous administration with ointment and percutaneous agents, and rectal administration with suppository. When administered orally, the LFA-1 inhibitors or the pharmaceutical compositions of the present invention can be prepared in the form of hard capsule, soft capsule, granule, powder, subtle granule, ball, troche, active ingredient sustained release agent, solution, suspension and the like. The preparation can be carried out easily by using general carrier of pharmaceutical field and by conventional means.

When the pharmaceutical compositions of the present invention are prepared in the form of oral administration, compositions for the preparation such as general carrier, for example diluent and excipient such as filler, expander, binder, disintegrating agents, surfactant, and lubricant, can be used. Examples of the compositions for the preparation include: excipient such as lactose, saccharose, sodium chloride, glucose, urea, starch, calcium carbonate, kaolin, crystalline cellulose, and silicic acid; binder such as water, ethanol, simple syrup, glucose solution, starch solution, gelatin solution, carboxymethylcellulose, shellac, methylcellulose, potassium phosphate, and polyvinyl pyrrolidone; disintegrating agents such as dried starch, sodium alginate, powdered agar, powdered laminaran, sodium bicarbonate, calcium carbonate, polyoxyethylene sorbitan fatty acid ester, sodium lauryl sulfate, monoglyceride stearate, starch, and lactose; disintegration inhibitor such as saccharose, stearic acid, cacao butter, and hydrogenated oil; absorption promoter such as quaternary ammonium salt and sodium lauryl sulfate; humectant such as glycerin and starch; adsorbent such as starch, lactose, kaolin, bentonite, and colloidal silicic acid; and lubricant such as purified talc and stearate. If needed, coloring agent, preservative, aroma chemical, flavor, sweetening agent and the like may be further formulated.

The form of parenteral administration of the pharmaceutical compositions of the present invention can be prepared by dissolving said polyamines alone or together with other compositions for the preparation in an appropriate solvent such as saline and phosphate buffer solution.

Effects and Metabolism of Polyamines

The polyamines used in the present invention are illustrated to take spermine and spermidine as an example, which are typical biogenic polyamines existing in a human body.

Polyamine is low molecular basic material containing nitrogen atoms and named from containing amine moieties in the molecule. Polyamine is contained in cells of almost all organisms including microorganisms, plants and animals at high concentration, i.e. mM unit (M represents mol/L). It is believed that polyamine plays a key role in cell proliferation or differentiation and intracellular signaling. It had been clarified that intracellular polyamine concentration in young individual which cell proliferation is active is high, and intracellular polyamine concentration is rapidly decreased by aging (20, 21). This is considered to be mainly because activity of intracellular enzyme for synthesizing polyamine described below is rapidly decreased with aging (22).

In contrast, not only polyamine concentration but also activity of the enzyme necessary to synthesize polyamine is high in self-proliferating cells. In fact, it has been clarified that polyamine concentration and activity of the polyamine synthetic enzyme in cancerous tissue actively dividing and proliferating in human patients with cancer exhibit high values as compared with those in its neighboring normal tissue.

For example, polyamines in the human body are mainly three kinds, spermine, spermidine and putrescine.

These polyamines are synthesized in the cell, and their synthetic pathways have been elucidated fully. First, ornithine is synthesized from arginine which is a kind of amino acid, by the effects of arginase. Ornithine is converted into putrescine by the effects of ornithine decarboxylase. From the production of the putrescine, the synthesis of polyamine starts.

Also, S-adenosylmethionine synthesized from methionine is decarboxylated by the effects of S-adenosylmethionine decarboxylase, and decarboxylated S-adenosylmethionine is produced.

By the effects of spermidine synthase (another name: spermine-spermidine synthase), the putrescine undergoes propylamine transfer from the decarboxylated S-adenosylmethionine, and spermidine is synthesized.

By the effects of spermine synthase (or spermine-spermidine synthase), spermidine undergoes propylamine transfer from the decarboxylated S-adenosylmethionine, and spermine is synthesized.

Unnecessary spermine and spermidine accumulated in the cell are acetylated by the effects of acetylCoA.

In other words, the spermine is acetylated and converted into N-acetylspermine by the effects of AcetylCoA, and further converted into spermidine by the effects of polyamine oxidase. Similarly, spermidine is acetylated and converted into N-acetylspermidine by the effects of acetylCoA, and further converted into putrescine by the effects of polyamine oxidase.

In this way, spermine and spermidine are synthesized and decomposed in the cell by the effects of synthetic enzyme and the effects of catabolic enzyme, and the intracellular concentrations of spermine and spermidine are adjusted.

Therefore, when any of the enzymes associated with the syntheses of spermine and spermidine in the cell are inhibited, it is considered that since spermine and spermidine are not synthesized and are exhausted in the cell, decrease of their concentrations is caused to influence cell growth and metabolism.

However, in the experiment using animals with transplanted cancerous tissue having autonomous and active synthesis of polyamines, it has been clarified that the polyamines in the cancerous tissue are not exhausted only by administering an inhibitor of polyamine synthetic enzyme to the animal and inhibiting the effects of the polyamine synthetic enzyme, and the cancer cells continue to proliferate. On the contrary, it has been known that administration of polyamine-free diet to this animal decreases polyamines in the cancer cell and downsizes the cancerous tissue. Namely, it has been clarified that the exhaustion of polyamines in the cell requires not only inhibiting the synthesis thereof in the cell but also cutting off supply of polyamines from the outside of the cell.

For example, it has been clarified that when a rat is fed with diet mixed with isotope-labeled polyamines, the polyamines in the diet are quickly absorbed from intestine and move into cells of each tissue in the body in a short time. Because many of putrescines among polyamines contained in foods are degraded during this process by diamine oxidase existing in intestine, the putrescines utilized in the body are merely 20 to 30% of those administered. However, spermine and spermidine in foods are absorbed in their original forms, and 95% or more of those orally administered move into various tissues and organs in the body. Alternatively, it has been clarified in the animal experiment that when polyamines are administered in a parenteral manner such as injection, spermine and spermidine in their original forms quickly move into each tissue in the body.

Polyamine concentration in blood as well as excretion of polyamines into urine is increased in diseases which actively produce polyamines. The excretion of polyamines into urine requires transporting the polyamines to kidney though blood flow. However, since polyamines are not present in plasma, it is considered that blood cells are responsible for the transportation of the polyamines in the body.

For example, according to the report of Bardocz et al., 85 to 96% of administered spermine and spermidine are absorbed from intestine. Moreover, 72 to 82% of molecules of the absorbed spermine and spermine extend to the entire body. However, many of putrescines are degraded, and only 29 to 39% of them are absorbed (23).

Considering the fact that polyamines ingested orally or administered intraperitoneally move very quickly in their undecomposed and original molecular forms into tissue in the body, it is evident that blood plays an important role for polyamines taken into the body. Moreover, it has been clarified that in human blood, polyamines are not detected in serum and are contained in high concentration in blood cells (erythrocyte, leukocyte, lymphocyte, and monocyte).

For example, according to the report of Cohen et al., polyamines are hardly detected in human plasma, and most of polyamines detected in blood are contained in erythrocyte. This is because the number of erythrocyte is large, in spite of low polyamine concentration in the erythrocyte. In lymphocyte, spermidine and spermine are contained in concentrations 100 times and 400 times, respectively, as much as their concentrations in erythrocyte. In erythrocyte, spermidine is contained in concentration higher than that of spermine. Meanwhile, in lymphocyte and granular leukocyte (granulocyte), spermine is contained in concentration higher than that of spermidine (24).

It has been clarified that polyamine concentration in the blood cells is increased in patients with diseases such as actively synthesizing polyamines in the body (25).

It has been clarified that the amount of polyamines produced in the body is decreased with aging, and polyamine concentration in peripheral blood is also decreased when polyamine concentration in the cell is decreased (26, 27, 28).

Thus, it is considered that polyamines move in the body by cells in blood (erythrocyte, leukocyte, lymphocyte, monocyte, and macrophage).

The extent to which polyamines contained in foods influence polyamine concentration in human peripheral blood monocyte is not clear. However, in the animal experiment, it has been clarified that polyamine concentration in foods influences polyamine concentration in immunocytes such as peripheral blood monocyte (lymphocyte, monocyte, and macrophage) (29).

As is evident from these descriptions, intracellular polyamine concentration is adjusted by synthesis and decomposition of polyamines in the cell, uptake thereof from the outside of the cell, and excretion thereof to the outside of the cell. Spermidine and spermine ingested orally have a significant impact on the concentration and construction of polyamines in the cells (including peripheral blood monocyte (lymphocyte, monocyte, and macrophage)) in the body.

Various concentrations of polyamines are contained in foods. Polyamine concentration and component ratio of polyamines (ratio of contents of spermine, spermidine, and putrescine) differ largely from food to food. Particularly, the contents of spermine and spermidine, which are absorbed in their original molecular forms and taken up into the cell, differ largely depending on food types. Spermine and spermidine are contained in high concentration in some beans such as soybean and green pea and mushrooms among natural foods. Large amounts of spermine and spermidine are also contained in fermented food products such as cheese and yogurt among processed foodstuffs (30). In other words, the type and amount of polyamines (particularly, spermine and spermidine) ingested by humans in a day differ largely depending on differences of eating habits in the regions where they live.

Possible methods for increasing intracellular spermine and spermidine concentrations include: a method comprising administering putrescine, ornithine, arginine, methionine, and S-adenosylmethionine serving as raw materials of spermine and spermidine; a method comprising activating enzymes necessary for intracellular synthesis of spermidine and spermine (e.g., ornithine decarboxylase, S-adenosylmethionine decarboxylase, spermidine synthase, spermine synthase, and spermine-spermidine synthase) and promoting synthesis of spermine and spermidine; a method comprising inhibiting the activity of enzymes which decompose the synthesized spermine and spermidine in the cell; and a method comprising orally or parenterally administering spermine and spermidine to allow them to be directly taken up form the outside of the body into the cell. Particularly, the method comprising orally or parenterally administering spermine and spermidine is simple and is capable of effectively increasing intracellular spermine and spermidine concentrations. Moreover, previous studies have also elucidated the daily intake of polyamines in human and the amounts of spermine and spermidine necessary to express acute toxicity.

For decreasing intracellular spermine and spermidine concentrations, the reverse effects of the methods described above may be employed. Namely, it is possible to decrease intracellular spermine and spermidine concentrations by a method comprising cutting off supply of putrescine, ornithine, arginine, methionine, S-adenosylmethionine and the like serving as substances necessary for synthesis of spermine and spermidine; a method comprising inhibiting the effects of enzymes necessary for synthesis of spermidine and spermine (e.g., ornithine decarboxylase, S-adenosylmethionine decarboxylase, spermidine synthase, spermine synthase, and spermine-spermidine synthase); a method comprising activating acetylCoA and polyamine oxidase having the effects of decomposing spermine and spermidine and promoting the decomposition of spermine and spermidine; and a method comprising cutting off oral or parenteral supply of spermine and spermidine from the outside of the body.

As described above, biogenic polyamines are inherently present in organisms, and the intracellular concentration thereof can be adjusted. However, heretofore, there has been no idea that polyamine concentration in an organism is adjusted in uses for inhibiting the expression of CD11a and CD18 and inhibiting the function of LFA-1 composed of both of them, as in the present invention. Namely, although a case of use of polyamines for the purpose of treating individual diseases has been reported previously, a phenomenon in which the function of LFA-1 is directly inhibited is a novel finding gained by the present inventor. A pharmaceutical composition for the treatment and a method for the treatment

A pharmaceutical composition containing the LFA-1 inhibitor of the present invention has an effect on the treatment and inhibition of diseases such as arteriosclerosis, autoimmune disease, allergy, ischemic reperfusion injury, and diabetic retinopathy, via the expression level of LFA-1 and the strong action of inhibiting LFA-1. The improvement, treatment, and inhibition effects on their symptoms are illustrated below.

Treatment and Inhibition of Arteriosclerosis

A pharmaceutical composition for the treatment of arteriosclerosis containing said LFA-1 inhibitor of the present invention and a method for the treatment and the inhibition of arteriosclerosis using the pharmaceutical composition are described. A preparation form, a method for the preparation, and a method for the administration of the pharmaceutical composition are the same as said LFA-1 inhibitor. A dose for the treatment of the disease that occurred is 0.02-20 mg/kg of body weight per day, preferably 0.05-10 mg/kg of body weight per day. In the treatment of arteriosclerosis, the polyamine is administered daily. Preferably, a dose for the inhibition of onset and progression of arteriosclerosis is 0.05-4 mg/kg of body weight per day.

Next, the inhibition of onset and progression of arteriosclerosis and the improvement of the symptoms by the LFA-1 inhibitor, which inhibits the expression of CD11a and CD18 and inhibits the function of LFA-1, are described based on documents.

Mine et al. have revealed that pravastatin which is a drug of hyperlipemia and also serves as an LFA-1 inhibitor inhibits arteriosclerosis of coronary artery (nutritional blood vessel of heart) of a human patient after cardiac transplantation (31).

Weitz-Schmidt et al. have revealed that a statin-based therapeutic agent of hyperlipemia inhibits arteriosclerosis, and its mechanism works through the inhibition of LFA-1 by the statin-based therapeutic agent (32).

Kallen et al. have revealed that lovastatin which is a therapeutic agent of hyperlipemia blocks adhesion between LFA-1 and ICAM-1 and inhibits arteriosclerosis (33).

Kawakami et al. have revealed that atorvastatin which is a therapeutic agent of hyperlipemia inhibits monocyte adhesion to vascular endothelial cell, and have indicated that this is the mechanism of arteriosclerosis inhibition (34).

Mine et al. have revealed that adhesion of cells via LFA-1 is important for onset of arteriosclerosis (35).

Nie et al. have indicated that in the experiment using rats, when adhesion of LFA-1 and ICAM-1 is inhibited by using LFA-1 or ICAM-1 antibodies, monocyte adhesion to vascular endothelium is decreased, and have revealed that the adhesion of LFA-1 and ICAM-1 plays a key role in onset of arteriosclerosis (36).

Suzuki et al. have indicated that in the experiment using mice, when adhesion of LFA-1 and ICAM-1 is inhibited even for a short time, progression of arteriosclerosis of transplanted heart is inhibited (37).

Russell et al. have revealed that in the experiment using mice of heart transplantation model, when adhesion of LFA-1 and ICAM-1 is inhibited by using LFA-1 or ICAM-1 antibodies, arteriosclerosis of blood vessels of the transplanted heart is inhibited (38).

From their knowledge, it will be understood to one skilled in the art that a pharmaceutical composition containing LFA-1 inhibitors has an effect on the treatment and inhibition of arteriosclerosis. Treatment and inhibition of autoimmune disease

A pharmaceutical composition for the treatment of autoimmune disease containing said LFA-1 inhibitor of the present invention and a method for the treatment and the inhibition of autoimmune disease using the pharmaceutical composition are described. A preparation form, a method for the preparation, and a method for the administration of the pharmaceutical composition are the same as said LFA-1 inhibitor. When used in the treatment of autoimmune disease, said polyamine is administered at 0.02 to 20 mg/kg of body weight per day, preferably 0.05 to 10 mg/kg of body weight per day. For preventing onset of autoimmune disease, preferably, said polyamine is administered at 0.05 to 4 mg/kg of body weight per day for consecutive days.

Next, the inhibition of autoimmune disease and the improvement of the symptoms by the LFA-1 inhibitor, which inhibits the expression of CD11a and CD18 and inhibits the function of LFA-1, are described based on documents.

Examples of autoimmune disease of interest of treatment with the pharmaceutical composition of the present invention include the following: psoriasis, type I diabetes (insulin-dependent diabetes mellitus), Graves' disease (Basedow disease), Hashimoto disease, autoimmune arthritis (Lyme arthritis, chronic rheumatoid arthritis), autoimmune cerebrospinal peripheral neuritis or degeneration, Sjogren's syndrome, uveitis, retinitis, degeneration, autoimmune renal disease (glomerulonephritis and the like), inflammatory bowel disease (Crohn disease, ulcerative colitis and the like), and primary cholangitis.

Next, knowledge on the relationship between the inhibition of LFA-1 and autoimmune disease is described.

Papp et al. have reported clinical examples that human psoriasis is improved by intravenous administration of a CD11a antibody (39).

Gottlieb et al. have reported clinical examples that psoriasis is improved by administration of efalizumab which is a CD11a antibody (40, 41).

Dedrick et al. have reported clinical examples that human psoriasis is improved by administration of efalizumab which is a CD11a antibody (42).

Zeigler et al. have reported that when human psoriasis tissue is transplanted to immunodeficient mice, the human psoriasis is improved by administration of a CD11a antibody to the mice (43).

Mysliwiec et al. have indicated that enhancement of fluorescence intensity of CD11a positive cell in blood of peripheral blood monocyte of a patient with type I diabetes (insulin-dependent diabetes mellitus) and a patient with high risk of onset thereof is proportional to a detection level of autoantibody against langerhans islet (insulin-secreting cell), and have revealed that LFA-1 has an important role in onset of type I diabetes (44).

Moriyama et al. have reported that onset of diabetes is inhibited by administration of an LFA-1 antibody to diabetic model of mice with pathology similar to human type I diabetes (insulin-dependent diabetes mellitus) (45).

Herold et al. have reported that onset of type I diabetes (insulin-dependent diabetes mellitus) is considered to be concerned in adhesion between LFA-1 of lymphocyte and ICAM-1 of insulin-secreting cell, and inflammation occurring in insulin-secreting cell is inhibited by using antibodies to LFA-1 in the experiment using mice with similar pathology (46).

Hasegawa et al; have reported that onset of diabetes can be prevented by administration of LFA-1 antibodies to mice of diabetic model corresponding to human type I diabetes (insulin-dependent diabetes mellitus) (47).

Kretowski et al. have reported that expression frequency of LFA-1 of peripheral blood monocyte in patients with type I diabetes (insulin-dependent diabetes mellitus) and Graves' disease (Basedow disease) and average fluorescence intensity of the positive cell are enhanced (48).

Guerin et al. have reported that the number of LFA-1 positive cell is increased in peripheral blood lymphocyte of patients (30 people) with Graves' disease (Basedow disease), and the number of the positive cell is decreased as the symptoms is improved by treatment, and have also reported that LFA-1 is involved in the enhancement of function of the thyroid gland (49).

Arao et al. have reported that LFA-1 positive lymphocyte is infiltrated in thyroid gland tissues of Graves' disease (Basedow disease), and thyroid gland cell proliferation starts by adhesion of thyroid gland cells and the lymphocytes, and have also reported that since LFA-1 antibodies can inhibit adhesion of peripheral blood monocytes and lymphocytes in thyroid gland to thyroid gland cells, LFA-1 has an important role in onset of Graves' disease (50).

Bagnasco et al. have reported that a number of lymphocytes of thyroid gland of Hashimoto disease (autoimmune disease of thyroid gland) are LFA-1 positive (51).

Marazuela et al. have reported that since tissues of thyroid gland of Graves' disease (Basedow disease), which is autoimmune disease, have a number of LFA-1 positive lymphocytes and tissues of thyroid gland of Hashimoto disease have a number of ICAM-1 positive cells, adhesion and reaction of LFA-1 of lymphocyte and ICAM-1 of thyroid gland cell are the important onset factor in the autoimmune disease of thyroid gland (52).

Steere et al have reported that LFA-1 has the structure moiety similar to antigen of offending bacteria of Lyme arthritis, and may have a central role of the pathology (53).

Gross et al. have reported that LFA-1 molecules may be the target of autoimmunity in pathology of Lyme arthritis which is special arthritis (54).

Birner et al. have reported that infiltration of lymphocytes to joints is increased in arthritis rheumatica, and by administration of LFA-1 antibodies to rat with similar disease, the symptoms of the arthritis are reduced (55).

Gordon et al. have reported that by using anti-CD11a antibody to rat models with disease similar to human neurodegenerative disease called multiple sclerosis, the progression and the severity of the disease are reduced (56).

Willenborg et al. have reported that by using anti-CD11a antibody to rat models with disease similar to human neurodegenerative disease called multiple sclerosis, the progression and the severity of the disease are reduced (57).

Archelos et al. have reported that symptoms of neuritis are improved by administration of anti-LFA-1 antibody to an animal model (rat) of human Guillain-Barre syndrome (58).

Inoue et al. have reported that progression of demyelinating disease is inhibited by administration of anti-LFA-1 antibody to mice with encephalomyelitis virus-induced demyelinating disease (exhibiting neurodegeneration) having pathology similar to human autoimmune cerebrospinal degenerative disease (59).

Kapsogeorgou et al. have reported that ICAM-1, which is LFA-1 ligand, is expressed highly in the salivary glands of patients with Sjogren's syndrome which is autoimmune disease (60).

Takahashi et al. have reported that ICAM-1, which is LFA-1 ligand, is expressed highly in the blood vessel in the salivary glands of mice with pathology similar to Sjogren's syndrome which is autoimmune disease, and LFA-1 is expressed highly in the lymphocytes infiltrated to lesion of the salivary glands (61).

Hayashi et al. have reported that when the disease of mice with pathology similar to Sjogren's syndrome is transplanted to other mice, the disease can be prevented by administration of anti-LFA-1 antibody (62).

Uchio et al. have reported onset of uveitis can be inhibited by administration of antibodies to LFA-1 and ICAM-1 to rats with the same disease as uveitis in human (63).

Whitcup et al. have reported onset of uveitis can be inhibited by administration of antibodies against LFA-1 and ICAM-1 to rats with the same disease as uveitis in human (64).

Ando et al. have reported that by administration of anti-LFA-1 antibodies to autoimmune retinitis in mice, the symptom is improved (65).

Nishikawa et al. have reported that progression of nephritis is inhibited by administration of anti-LFA-1 antibodies to rats with disease similar to human glomerulonephritis (66).

Kawasaki et al. have reported that by administration of anti-LFA-1 antibodies to rats with disease similar to human glomerulonephritis, the progression of the condition of the disease can be inhibited (67).

Kootstra et al have reported that the finding of nephritis is improved by administration of anti-LFA-1 antibodies to animal model of lupus nephritis which is autoimmune disease in human (68).

Taniguchi et al have reported that by administration of anti-ICAM-1 antibodies to disease models of rats with the disease similar to human inflammatory bowel disease (ulcerative colitis and Crohn disease) and inhibition of adhesion to intestinal mucosa cells of immunocytes expressing LFA-1, the symptom is improved (69).

Vainer et al have reported that expression of CD18 is enhanced in immunocytes infiltrated in intestinal mucosa of patients with ulcerative colitis, and expression of CD11a is enhanced in the cell of patients with Crohn disease (70).

Kimura et al have reported that by administration of anti-LFA-1 antibodies to the mice with the disease similar to human primary biliary cirrhosis, the symptom is improved (71).

Shiina et al. have reported that the number of cells strongly expressing LFA-1 in peripheral blood lymphocytes in patients with primary biliary cirrhosis which is autoimmune disease is larger than those in normal people (72).

As is clear from their knowledge, it will be understood to one skilled in the art that a pharmaceutical composition containing LFA-1 inhibitors has an effect on the treatment and inhibition of autoimmune disease.

Treatment and Inhibition of Allergy

A pharmaceutical composition for the treatment of allergy containing said LFA-1 inhibitor of the present invention and a method for the treatment and the inhibition of allergy using the pharmaceutical composition are described. A preparation form, a method for the preparation, and a method for the administration of the pharmaceutical composition are the same as said LFA-1 inhibitor. When used in the treatment of allergy that occurred, said polyamine is administered at 0.02 to 20 mg/kg of body weight per day, preferably 0.05 to 10 mg/kg of body weight per day. When used for the purpose of preventing onset of allergy, preferably, said polyamine is administered at 0.05 to 4 mg/kg of body weight per day for consecutive days.

Next, the inhibition of allergy and the improvement of the symptoms by the LFA-1 inhibitor, which inhibits the expression of CD11a and CD18 and inhibits the function of LFA-1, are described based on documents.

Pesce et al. have reported that LFA-1 is expressed highly on conjunctival epithelium cells of patients with allergic conjunctivitis (73).

Whitcup et al. have reported that by administration of anti-LFA-1 or anti-ICAM-1 antibodies to mice with allergic conjunctivitis, the symptom of the conjunctivitis is improved (74).

Tomita et al have reported that expression of CD11a in lymphocytes and monocytes of patients with atopic asthma is enhanced (75).

Asakura et al. have reported that by administration of anti-LFA-1 antibodies to rats with allergic rhinitis, the number of eosinophil infiltrated during onset of allergy is decreased, and the symptom is alleviated (76).

Rote et al. have reported that the symptom of Arthus reaction (one kind of allergic reaction) in rats can be reduced by administration of anti-LFA-1 antibodies (77).

Winquist et al. have reported that by administration of a small molecule produced for inhibition of LFA-1 to allergic dermatitis of animals, the symptom is improved (78).

Murayama et al. have reported that by administration of anti-LFA-1 antibodies to contact dermatitis of mice, the symptom is improved (78).

Hakugawa et al. have reported that in the experiment using mice, the allergy reaction is inhibited by administration of anti-LFA-1 antibodies to delayed allergy in skin (79).

Bloemen et al. have reported that when allergic asthma is induced in a mouse, the mouse has resistance to the asthma by administration of anti-LFA-1 antibodies (80).

Tanaka et al. have reported that by administration of anti-LFA-1 antibodies to a mouse, the reaction of IgE, which is one of immunoglobulins having important effects on immunoreaction of allergy to allergen, is decreased (81).

As is clear from their knowledge, it will be understood to one skilled in the art that a pharmaceutical composition containing LFA-1 inhibitors has an effect on the treatment and inhibition of allergy.

Treatment and Inhibition of Ischemic Reperfusion Injury

A pharmaceutical composition for the treatment of ischemic reperfusion injury containing said LFA-1 inhibitor of the present invention and a method for the treatment and the inhibition of ischemic reperfusion injury using the pharmaceutical composition are described. A preparation form, a method for the preparation, and a method for the administration of the pharmaceutical composition are the same as said LFA-1 inhibitor. In the treatment and inhibition of ischemic reperfusion injury in a patient developing cardiac infarction, angina pectoris, cerebral infarction, transient cerebral ischemic attack or the like, said polyamine is administered at 0.02 to 40 mg/kg of body weight per day, preferably 0.05 to 20 mg/kg of body weight per day.

Next, the inhibition of ischemic reperfusion injury and the improvement of the symptoms by the LFA-1 inhibitor, which inhibits the expression of CD11a and CD18 and inhibits the function of LFA-1, are described based on documents.

Ischemic reperfusion injury refers to tissue injury caused by reperfusion of blood after vascular obstruction to tissues of cardiac infarction, angina pectoris, cerebral infarction, transient cerebral ischemic attack, transplanted organs, and the like, in which blood flow was transiently blocked for a certain period.

Marubayashi et al have reported that blocking of blood flow of rat liver, followed by blood reflow damages the liver cell, but the damage of the tissues is improved by pre-administration of anti-LFA-1 antibodies and anti-ICAM-1 antibodies (82).

Tajra et al. have reported that blocking of renal blood vessel of rats, followed by blood reflow damages the kidney, but the renal damage is reduced by administration of anti-LFA-1 antibodies before the blood reflow (83).

Da Silva et al. have reported that when the kidney of monkeys is cooled and allowed to fall into an ischemic state, the renal damages attributed to subsequent blood reflow are reduced by administration of anti-LFA-1 antibodies (84).

Kelly et al. have reported that blocking of ambilateral renal blood flow of rats, followed by blood reflow causes renal functional damage, but the damage can be prevented by administration of anti-LFA-1 antibodies and anti-ICAM-1 antibodies (85).

Childs et al. have reported that inflammation cell infiltration in small bowel is caused by treatment that improves blood pressure after hemorrhagic shock in rats, but the reaction can be prevented by administration of anti-LFA antibodies (86).

DeMeester et al. have reported that when rat lung is extirpated and retransplanted, the lung graft survival is improved by administration of anti-ICAM-1 antibodies to the extirpated lung and administration of anti-LFA-1 antibodies and anti-ICAM-1 antibodies to recipient rats (87).

As is clear from their knowledge, it will be understood to one skilled in the art that a pharmaceutical composition containing LFA-1 inhibitors has an effect on the treatment and inhibition of ischemic reperfusion injury. Treatment and inhibition of diabetic retinopathy

A pharmaceutical composition for the treatment of diabetic retinopathy containing said LFA-1 inhibitor of the present invention and a method for the treatment and the inhibition of diabetic retinopathy using the pharmaceutical composition are described. A preparation form, a method for the preparation, and a method for the administration of the pharmaceutical composition are the same as said LFA-1 inhibitor. In the inhibition of onset or the treatment of diabetic retinopathy, said polyamine is administered at 0.02 to 20 mg/kg of body weight per day, preferably 0.05 to 10 mg/kg of body weight per day.

The inhibition of diabetic retinopathy and the improvement of the symptoms can be conducted by the LFA-1 inhibitor, which inhibits the expression of CD11a and CD18 and inhibits the function of LFA-1. Namely, Barouch et al. have indicated that when the functions of LFA-1 are inhibited by administration of antibodies of CD18 to diabetic rats, the number of leukocytes infiltrated in retina is decreased, and have reported that the antibodies have the effects of inhibiting the progression of diabetic retinopathy (88).

As is clear from their knowledge, it will be understood to one skilled in the art that a pharmaceutical composition containing LFA-1 inhibitors has an effect on the treatment and inhibition of diabetic retinopathy. Inhibition of rejection and method thereof.

A pharmaceutical composition containing said LFA-1 inhibitor of the present invention has inhibition effects on rejection in transplantation. A preparation form, a method for the preparation, and a method for the administration of the pharmaceutical composition are the same as said LFA-1 inhibitor. The inhibition of rejection of transplanted organs and tissues and the improvement of graft survival rate of the transplanted organs and tissues can be performed by the LFA-1 inhibitor, which inhibits the expression of CD11a and CD18 and inhibits the function of LFA-1. For inhibiting rejection in organ transplantation, said polyamine is administered at 0.02 to 20 mg/kg of body weight per day, preferably 0.05 to 10 mg/kg of body weight per day. When the pharmaceutical composition is used as a perfusate for perfusion of transplanted organs in order to inhibit rejection of the transplanted organs, the perfusate containing 1 μM to 10 mM of said polyamine, preferably 10 μM to 2 mM of said polyamine is used.

A method for the inhibition and inhibition of rejection using the pharmaceutical composition is described based on documents.

Kobashigawa et al. have found that pravastatin which is a drug of hyperlipemia and also serves as an LFA-1 inhibitor increases graft survival rate of transplanted heart in human patients of cardiac transplantation, and have reported this finding (89).

Dedrick et al. have reported that graft survival rate of transplanted kidney in humans can be increased by using antibodies to CD11a (90).

Werther et al. have reported that in the experiment using rhesus, graft survival rate of bone marrow transplantation can be increased by using antibodies to LFA-1 (91).

Pietersz et al. have reported that in the experiment using mice, rejection of a transplanted organ (heart) is inhibited by using antibodies to LFA-1 and ICAM-1 (92).

Ozer et al. have reported that in the experiment using rats, rejection of a transplanted organ (limb) is inhibited by using antibodies to LFA-1 and ICAM-1 (93).

Morikawa et al. have reported that in the experiment using mice, rejection of a transplanted organ (lung) is inhibited by using antibodies to LFA-1 (CD11a) (94).

Bowles et al. have reported that in the experiment using rats, rejection of a transplanted organ (small bowel) is inhibited by using antibodies to LFA-1 (95).

Grochowiecki et al. have reported that in the experiment using rats, rejection of a transplanted organ (pancreatic Langerhans cell (insulin-secreting cell), used for the treatment of diabetes) is inhibited by using antibodies to LFA-1 (96).

Bashuda et al. have reported that in the experiment using rats, rejection of a transplanted organ (heart) is inhibited over a long period by using antibodies to LFA-1 even for a short time (97).

Guerette et al. have reported that the effective treatment for human patients with Duchenne-Aran muscular atrophy is transplantation of muscular tissue, and in the experiment using mice, rejection of the transplanted muscular tissue is inhibited by administration of antibodies to LFA-1 in the muscular transplantation (98).

In a large number of transplantation experiments of animals other than findings listed herein, it has been reported that anti-LFA-1 antibody inhibits rejection of transplanted organs.

As is clear from their knowledge, it will be understood to one skilled in the art that a pharmaceutical composition containing LFA-1 inhibitors has an effect on the inhibition and inhibition of rejection of a transplant organ.

Hereinafter, Examples are described. These Examples are intended to describe in detail the contents of the present invention and do not limit the scope of protection of the present invention by any means.

All temperatures are Celsius temperatures (° C.). Abbreviations used respectively have the following meanings: (g): gram, (kg): kilogram, (mol): mole, (μmol): micromole, (mL): milliliter, (L): liter, (M): mol/L, (mp): melting point, (mm/Hg): pressure expressed as milliliter of mercury, and (bp): boiling point.

### EXAMPLES

### Example 1

### Preparation of Cells

In this Example, peripheral blood monocytes provided by volunteers were used.

Human peripheral blood was collected, and peripheral blood monocytes including lymphocyte, monocyte and the like were recovered from the collected blood by specific gravity centrifugation technique using SEPARATE-L (Muto Pure Chemicals Co. LTD., Tokyo, Japan). Next, the recovered peripheral blood monocytes were suspended in PRMI1640 culture medium (Sigma chemical co., St. Louis, USA) mixed with 10% human serum (Wako Pure Chemical Industries LTD., Osaka, Japan), 0.1% L-glutamine (Invitrogen Corp., CA, USA), and 0.01% penicillin-streptomycin (Invitrogen Corp., CA, USA) and cultured in the humidified air at 37° C. containing 5% carbonic acid gas. At the start of the culture, spermine (spermine tetrahydrochloride; ICN Biomedicals Inc., Ohio, USA), spermidine (spermidine trihydrochloride; ICN Biomedicals Inc., Ohio, USA), or putrescine (1,4-Butanediamine dihydrochloride; Wako Pure Chemical Industries LTD, Osaka, Japan) was added to the cell culture medium in the final concentration of 0 μM (micromole/liter), 100 μM, or 500 μM in the culture medium. Following culture for a given time, the peripheral blood monocytes were recovered from the culture medium and used in Examples 2, 4, 5, and 6.

For confirming that obtained experimental results were not derived from changes produced by the polyamines present in the culture medium and kept in contact with the cells for a long time, different peripheral blood monocytes were prepared. Namely, peripheral blood monocytes, which after cultured with the polyamines for 16 to 24 hours, were washed and further cultured in culture medium without polyamines for 48 to 56 hours, were used as the different peripheral blood monocytes in Examples 2, 4, 5, and 6 below.

### Example 2

### Detection of Cell Membrane Differentiation Antigen of Human Peripheral Blood Monocyte

The peripheral blood monocytes cultured for 16 to 80 hours by the method of Example 1 were collected from the cell culture plate so as not to damage the cells. The collected cells were washed with PBS (−) solution and then fixed using phosphate buffered saline without calcium chloride and without magnesium chloride (hereinafter, referred to as PBS (−); Invitrogen Corporation, GIBCO, Grand Island, N.Y., USA) containing 2% paraformaldehyde (Wako Pure Chemical Industries LTD, Osaka, Japan), so as not to achieve change in cell membrane antigen molecules on the cell surface of the peripheral blood monocytes. The peripheral blood monocytes were further washed, to which antibodies to the cell membrane antigen molecules were then added in an amount corresponding to 5 μL (microliter) per 500000 cells. The antibodies used here are CD2 (FITC label), CD4 (FITC), CD8 (PE label), CD11a (FITC), CD11b (PE), CD11c (PE), CD18 (FITC), CD31 (PE), CD49d (PE), CD49e (PE), CD54 (PE), CD62L (PE), CD95 (FITC), and VIA-PROBE (FITC). All of these antibodies are manufactured by PharMingen (A Becton Dickinson Company, San Jose, Calif., USA).

After each antibody was added to the cells and reacted in the dark for 20 minutes, positive rate and light intensity of the cell membrane differentiation antigen expressed in the cultured peripheral blood were measured with FACS analyzer (FACSCalibur; manufactured by Becton Dickinson) (Becton Dickinson Japan, Tokyo). The population of the peripheral blood monocytes was of interest in the measurement of florescence of the cell surface, and the measurement of all of the cultured cells was also conducted at the same time. Further, a negative control was used, and a measurement gate was set to obtain each antibody positive rate of the cells within the gate and average fluorescence intensity of the positive cells.

Experimental Results

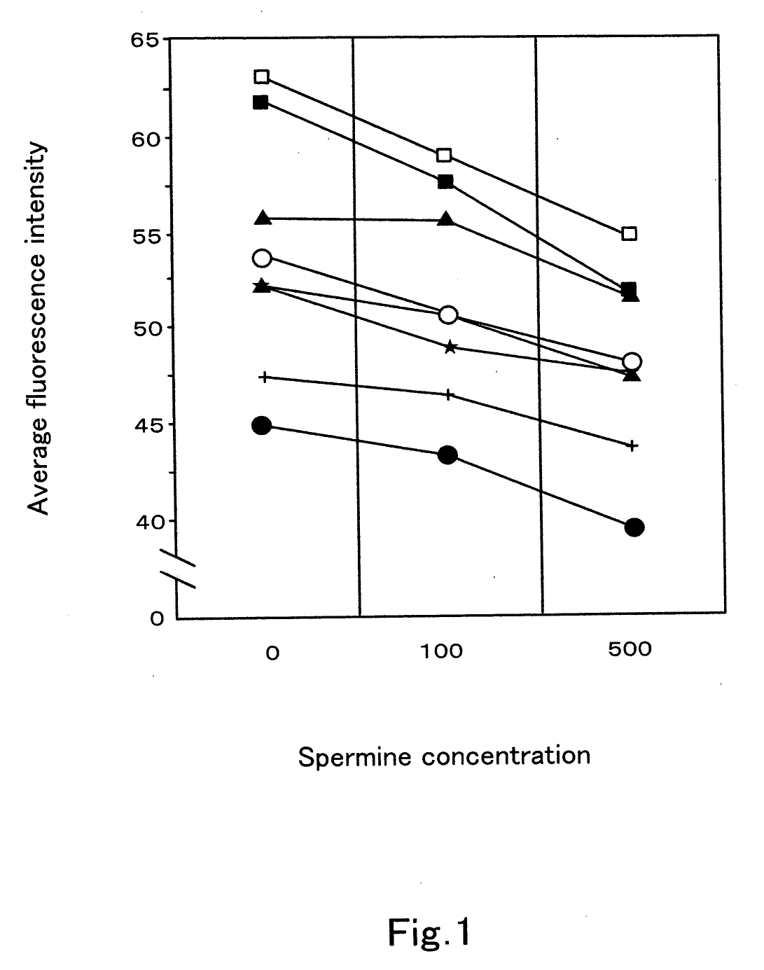

The average fluorescence intensity of CD11a and CD18 among the cell membrane surface antigens of the human peripheral blood monocytes (including lymphocyte, monocyte, macrophage, and natural killer (NK) cell) cultured in the cell culture medium mixed with spermine was inhibited. This inhibition got stronger with increase of spermine concentration, and concentration dependence was observed in the inhibition of fluorescence intensity of CD11a by spermine. Namely, as described in FIG. 1, the average fluorescence intensity of CD11a of the peripheral blood monocytes cultured with spermine for 70 to 80 hours was reduced as spermine concentration was increased. Each symbol in FIG. 1 represents an actual measurement value of change of average fluorescence intensity of CD11a in each individual blood.

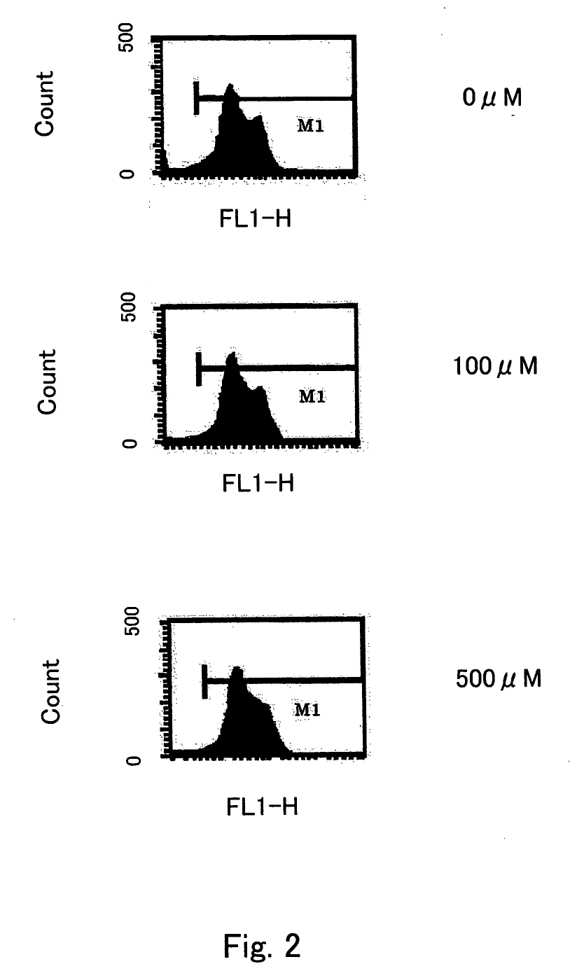

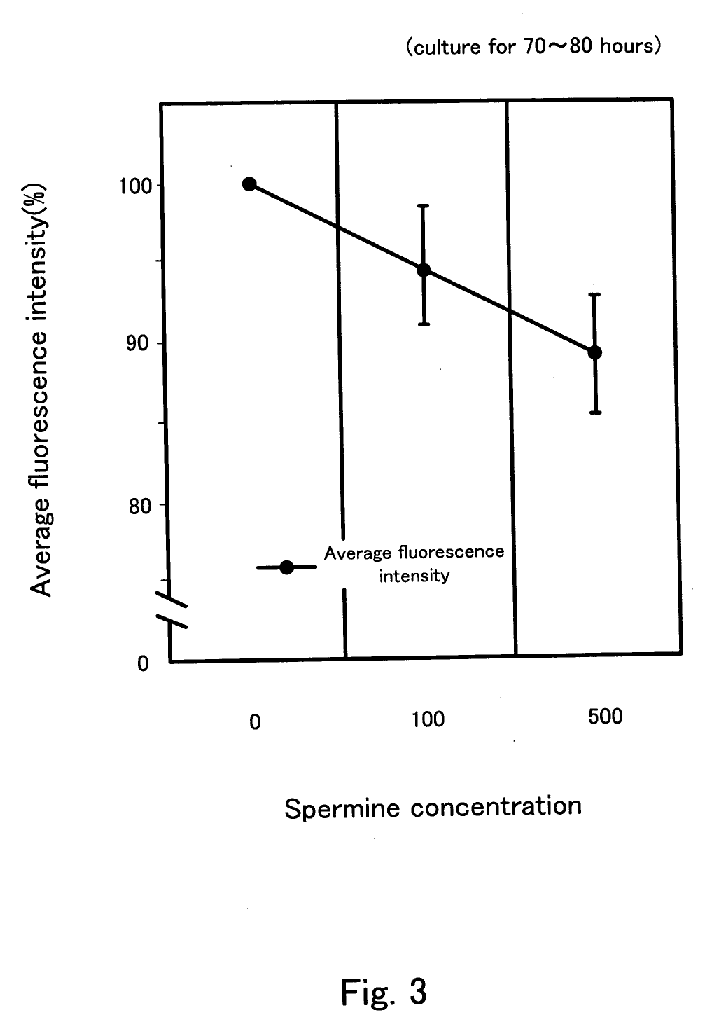

CD11a histogram is shown in FIG. 2. The horizontal axis of the histogram represents fluorescence intensity of the cells. This means that a cell expressing CD11a more strongly is located closer to the right on the horizontal axis. The vertical axis of the histogram represents the number of the cells. This means that a hill gets higher in the direction of the vertical axis as the number of the cells having the same fluorescence intensity gets larger. As seen from FIG. 2, a right-hand hill in the histogram is low for the cells supplemented with spermine, indicating that the number of the cells having strong fluorescent intensity is decreased. Likewise, the fluorescence intensity of CD18, which is a molecule composing LFA-1 together with CD11a, was also decreased by spermine (FIG. 3). As with CD11a, the average fluorescence intensity of CD18 was decreased with increase of spermine concentration.

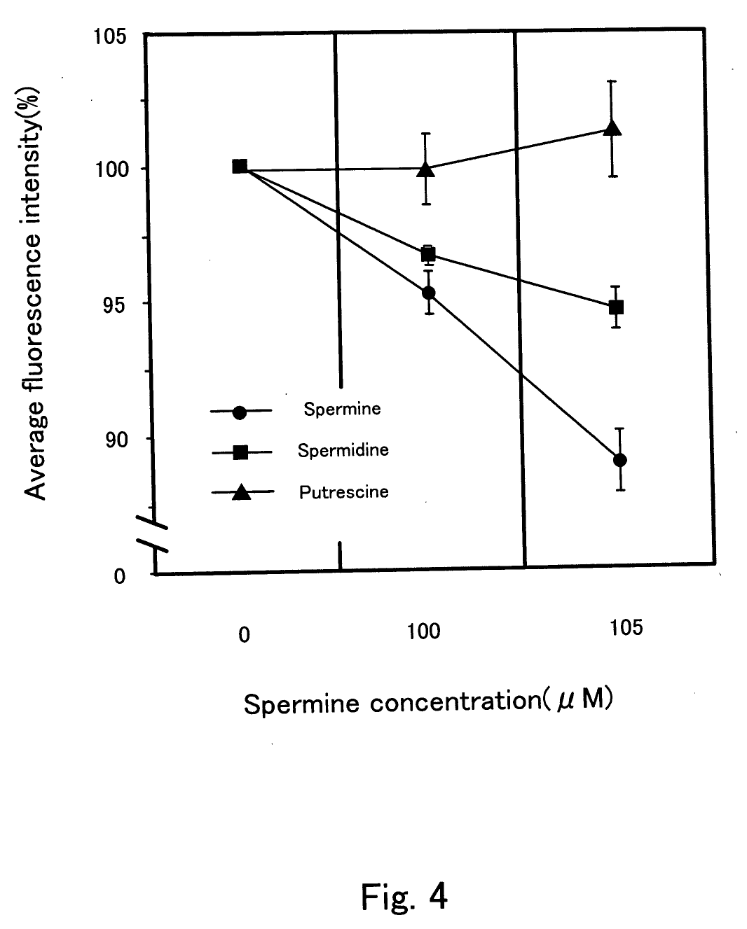

As shown in FIG. 4, the average fluorescence intensity of CD11a of the human peripheral blood monocytes cultured in the cell culture medium mixed with spermidine was inhibited dependently on spermidine concentration. This decreasing effect was strongest in spermine, followed by spermidine, while the decreasing effect exhibited by putrescine was not evident.

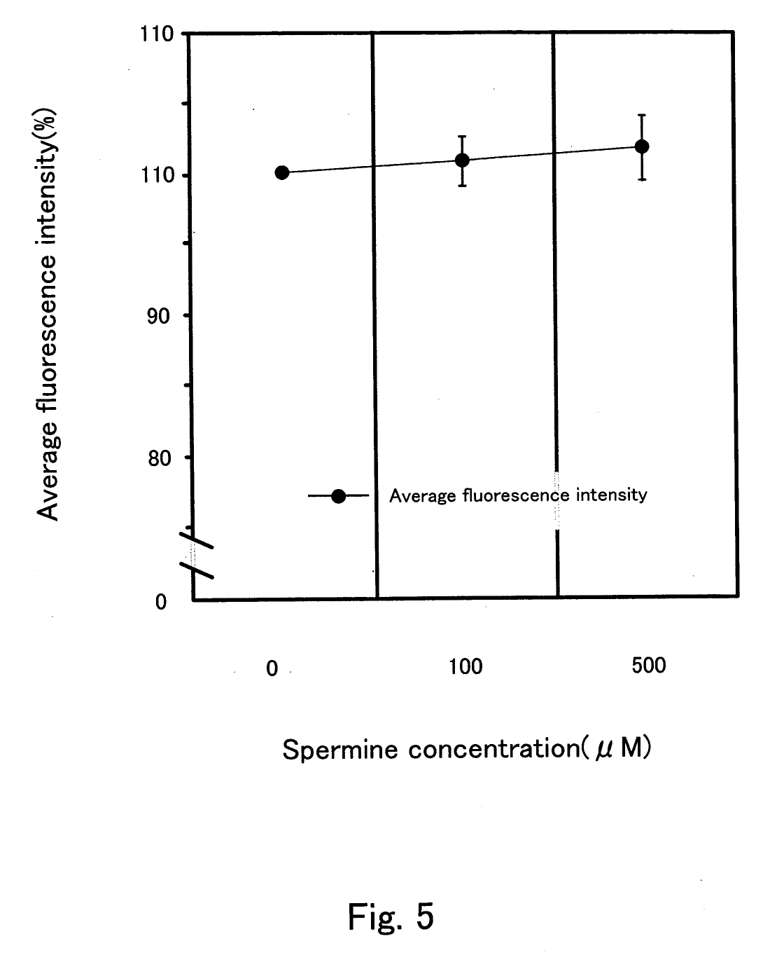

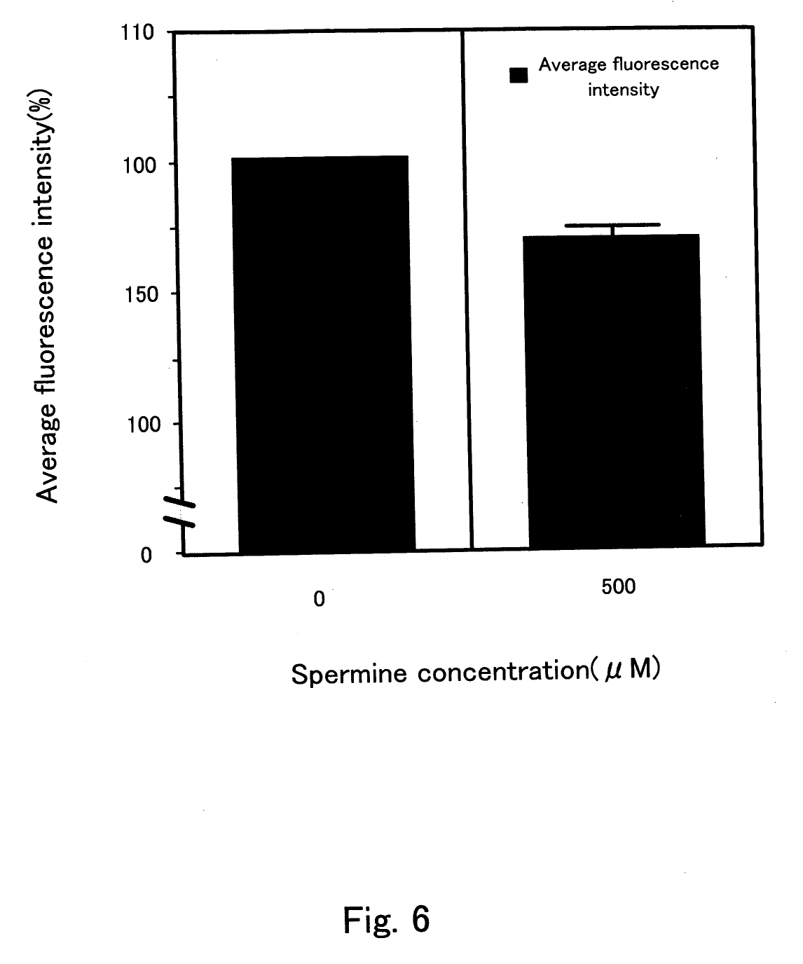

As shown in FIG. 5, the decreasing effect by spermine on the average fluorescence intensity of CD11a was not observed in the cells cultured for 20 to 26 hours. However, as shown in FIG. 6, decrease of the average fluorescence intensity of CD11a in the cells which after cultured in the culture medium supplemented with spermine for 16 to 24 hours, were washed and then cultured in the culture medium without spermine for 48 to 56 hours was observed as in the cells cultured with spermine for approximately 70 to 80 hours. Namely, it is suggested that the decrease of the average fluorescence intensity of CD11a is not derived from changes produced by the contact for a long time between high concentration of polyamines and the peripheral blood monocytes, that is, is not the direct effect by the extracellular polyamines on the CD11a molecule on the cell surface from the outside of the cells.

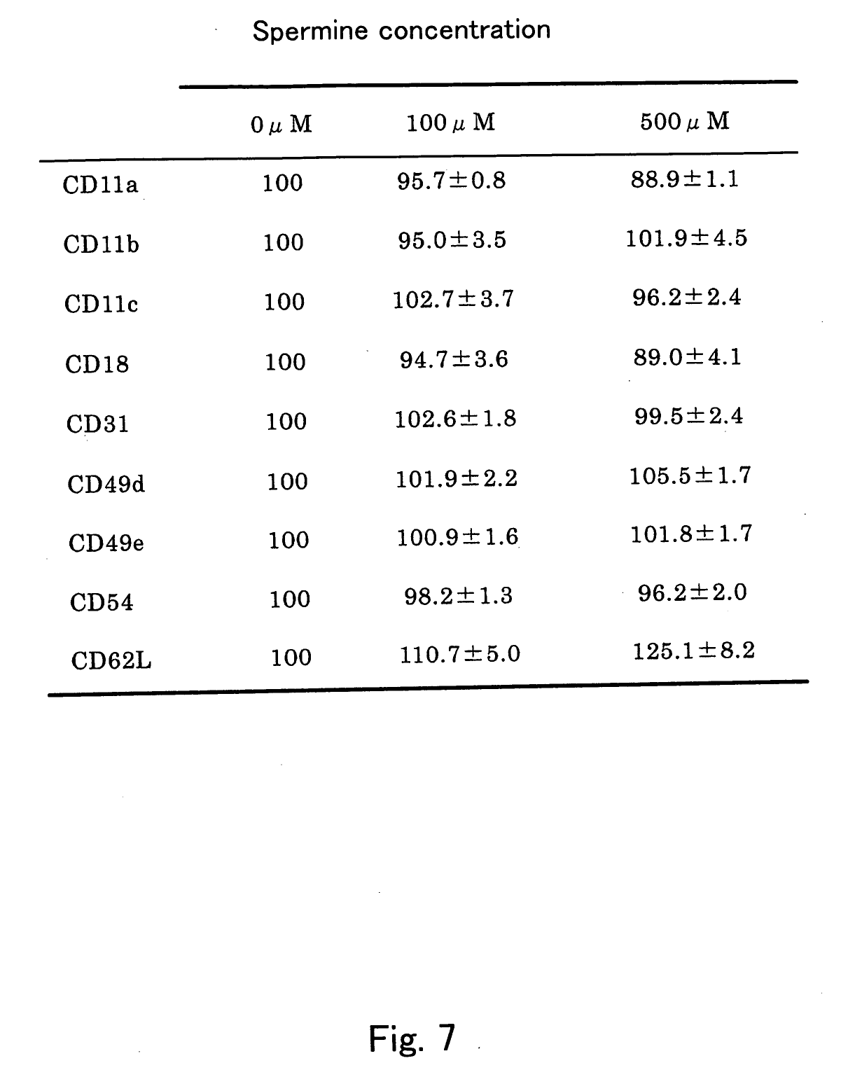

As shown in FIG. 7, the average fluorescence intensity of adhesion molecules (CD11b, CD11c, CD31, CD49d, CD49e, CD54, and CD62L) except CD11a and CD18 was not decreased by spermine and spermidine. The average fluorescence intensity of CD62L was obviously increased dependently on spermine concentration.

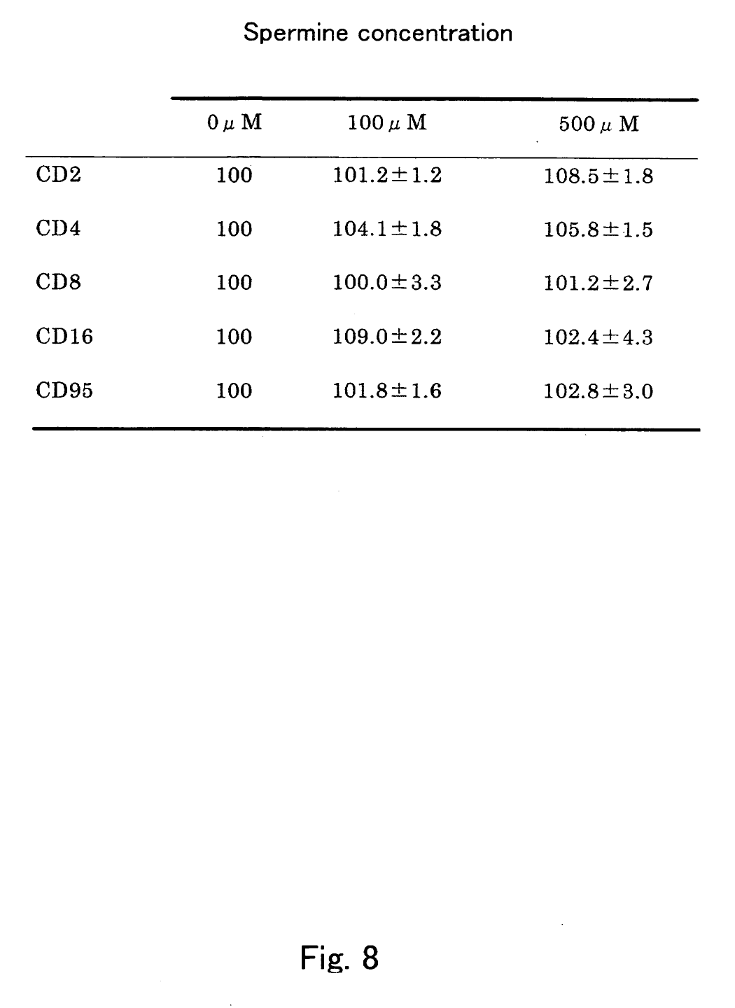

As shown in FIG. 8, no decrease of average fluorescence intensity was observed in the cell membrane differentiation antigens shown to be important for cell functions, other than the adhesion molecules.

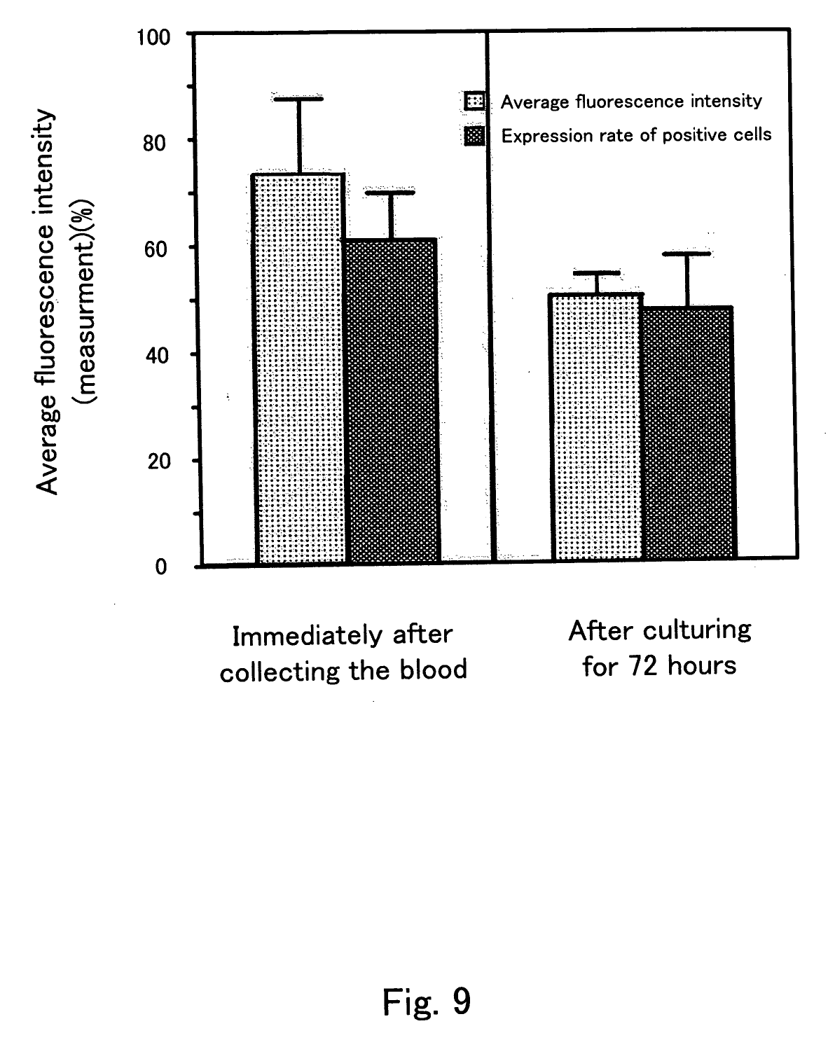

It is also possible that the change of CD11a depending on spermine resulted from increase of expression of cells strongly expressing CD11a (referred to as CD11a bright) on the surface of the peripheral blood monocytes, by stimulation of the culture plate during the culture of the peripheral blood monocytes. Namely, it is also conceivable that expression of CD11a bright was supposed to be increased by stimulation of the culture plate but was not increased by the presence of spermine or spermidine. However, as shown in FIG. 9, when comparison before and after cell culture was made on average fluorescence intensity of CD11a and expression rate of CD11a positive cell, it was clarified that the fluorescence intensity and the expression rate after the culture were decreased more than those before the culture. Thus, the hypothesis was denied that expression intensity of CD11a was supposed to be increased by the stimulation of the peripheral blood monocytes with the culture plate but this increase was inhibited by the presence of spermine or spermidine.

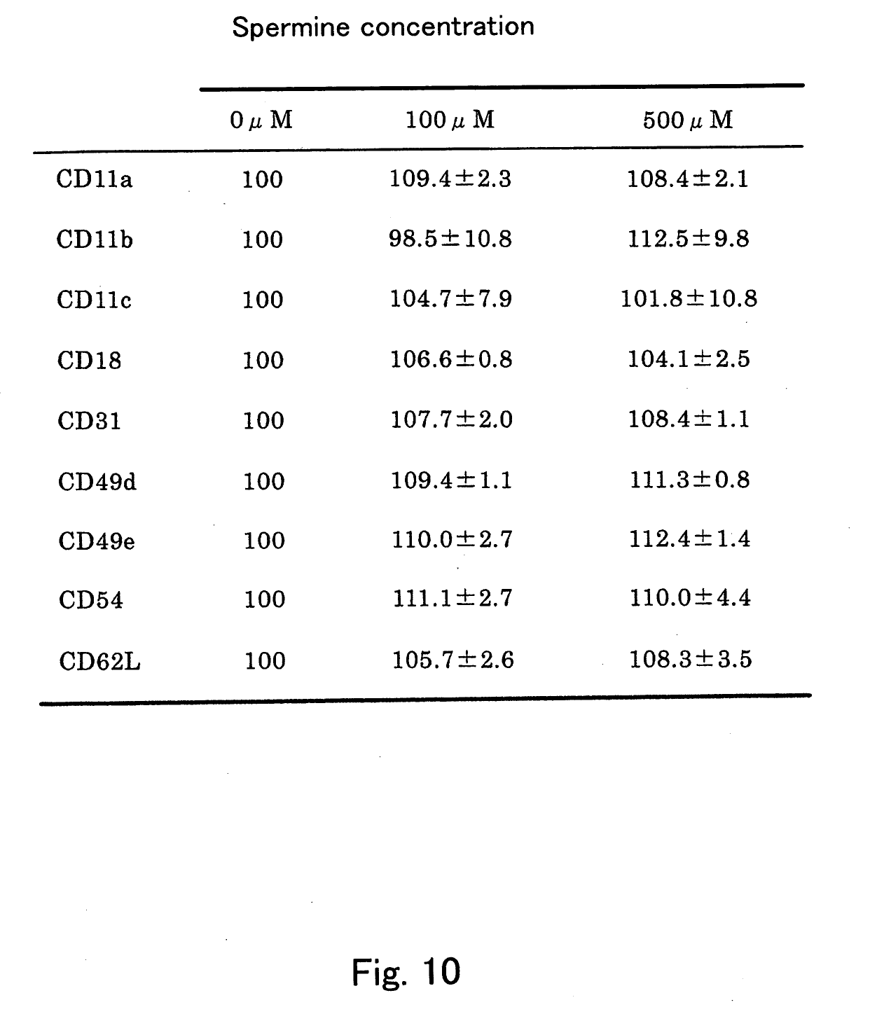

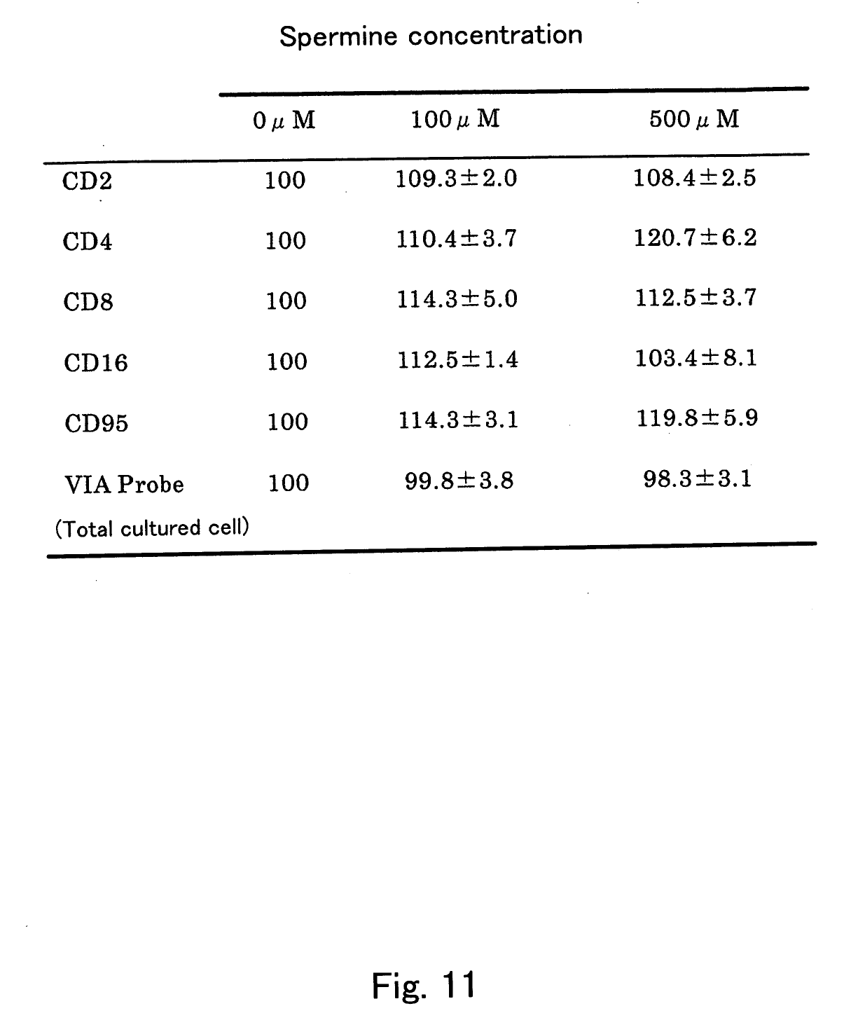

As described above, it was clarified that the average fluorescence intensity of CD11a and CD18 of the peripheral blood monocytes cultured with spermine and spermidine is decreased. However, as shown in FIG. 10, the rate to the total cells of the cells with expression of CD11a or CD18 was not decreased in the peripheral blood monocytes cultured with spermine. This was also observed in the peripheral blood monocytes cultured with spermidine. Although the number itself of the cell expressing CD11a on the cell surface was increased by spermine (and spermidine) as shown in FIG. 10, the average fluorescence intensity was decreased as shown in FIGS. 1, 4, and 6. Therefore, it is clear, as shown in FIG. 2, that the cells strongly expressing CD11a were decreased. Similarly, as shown in FIG. 11, the expression positive cells of the cell membrane differentiation antigens having an important role in cell functions, other than the adhesion molecules were not decreased. VIA-Probe is taken up into dead cells but not into live cells and as such, can be used to investigate the number of the dead cells. As shown in FIG. 11, even when the cells were cultured with spermine having a concentration that most strongly decreased average fluorescence intensity of CD11a and CD18, the dead cells were not increased. Thus, it is clear that spermine and spermidine exhibit no cellular cytotoxicity and selectively inhibit only CD11a and CD18 among the cell surface antigens.

When viral infection was found immediately after collection of blood or microbial infection was caused during culture, that is, when cytokine or the like was produced from the cultured peripheral blood monocytes, totally different experimental results were obtained.

It has already been clarified that the polyamines have an effect of inhibiting cytokine production (99). When cultured cells are infected, cytokine is produced from the cultured cells by stimulation of virus and bacteria etc, but the production of the cytokine is inhibited by the polyamines. Meanwhile, expression of any of adhesion molecules is increased by the production of the cytokine (100, 101, 102, 103).

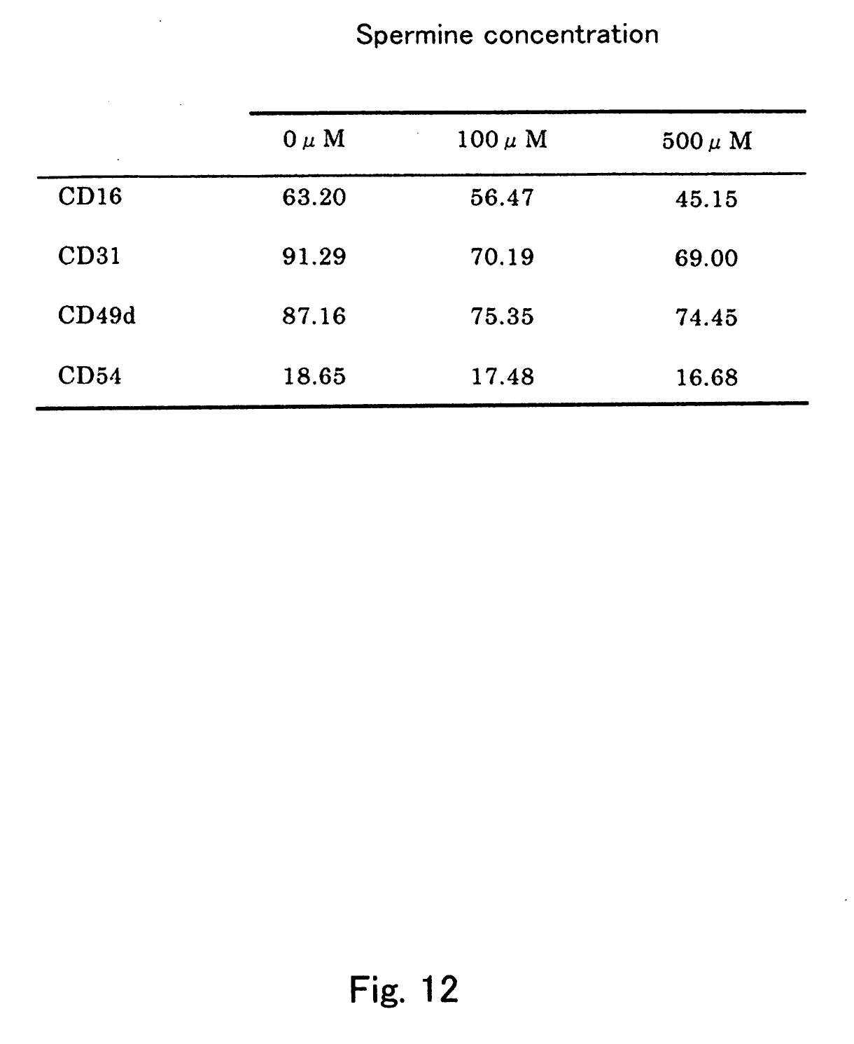

When cytokine is produced from the cultured peripheral blood monocytes, cytokine production should be inhibited with increase of polyamine concentration, and expression of the adhesion molecules whose expression is increased by the cytokine production should be decreased apparently. Data of an experiment that accidentally caused viral or microbial infection is shown in FIG. 12. As seen from FIG. 12, average fluorescence intensity of CD16, CD31, CD49d, and CD54 previously reported to have expression increased by cytokine is low in the cells cultured in the culture medium supplemented with the polyamines. This change is totally different from the change of the cell membrane differentiation antigens by the polyamines in the absence of infection. Thus, it is evident that the inhibition of average fluorescence intensity of CD11a and CD18 by the polyamines is not the inhibition by the polyamines of increase of the average fluorescence intensity by physical and chemical stimulation attributed to cell culture.

### Example 3

### Inhibition of Peripheral Blood Monocyte Adhesion to Culture Plate by Polyamines

When cells are cultured, cells cultured in a cell culture plate adhere onto the culture plate. It has been found that CD11a and CD11c of adhesion molecules of the cell surface are important for the adhesion of cells on a plate (104).

Peripheral blood monocytes were cultured on a 96-well cell culture plate using the culture medium described above. After spermine, spermidine, or putrescine was added in varying concentrations, the resulting cells were cultured for a given time. Following the culture, the cell culture plate was washed three times with PBS (−) solution to remove the cells floating in the culture medium. As a result, there remained only the cells adhering on the inside bottom of the cell culture plate.

Further, for the purpose of underlining difference of adhesion, the cells were cultured with varying concentrations of spermine for 70 to 82 hours. Then, the cell culture plate was inverted and centrifuged in a centrifuge at 500 rpm for 5 minutes to exfoliate the cells weakly adhering on the inside bottom of the culture plate. The culture supernatant was removed. As a result, there remained only the cells firmly adhering on the inside bottom of the cell culture plate, as described above.

Cell culture medium was freshly added to these cell culture plates, followed by culture for 1 hour. Thiazolyl blue (MTT) (C₁₈H₁₆N₅SBr) (Sigma. St. Louis, USA) was added in the final concentration of 0.35 mg/mL, and the cells were cultured at 37° C. for 2 to 4 hours until the cells were stained. MTT is taken up into cells, and the dye is discolored only in live cells. Therefore, the amount of the live cells existing can be confirmed from the amount of the dye. This means that the larger the amount of the dye is, the higher the number of the cells and the cell activity are. After the cells were sufficiently stained with MTT, the culture supernatant was removed by aspiration. The cells were dissolved by addition of 100 μl of cell lysis solution (2-propanol (isopropanol; Wako Pure Chemical Industries LTD, Osaka, Japan) mixed with 12 M (mol/L) hydrochloric acid (HCl; Wako Pure Chemical Industries LTD, Osaka, Japan)), to recover the dye in the cells. Absorbance in the cell culture plate was measured at 2 absorbance wavelengths of 570 nm and 690 nm using an absorbance reader (Titertek Multiskan MCC/340, Labsystems, Flow Laboratories Inc., USA).

For confirming that spermine and spermidine exhibit no cytotoxicity to the cultured cells, peripheral blood monocytes cultured simultaneously with the culture described above under the same conditions were used to conduct an experiment for investigating the number and activity of the total cultured cells. Namely, MTT was added to the culture supernatant of the peripheral blood monocytes cultured for a given time in a culture plate supplemented with varying concentrations of spermine, spermidine, or putrescine, and the cells were cultured at 37° C. for 2 to 4 hours until the cells were sufficiently stained. After the cells were sufficiently stained, the culture plate was centrifuged in a centrifuge at 1000 rpm for 10 minutes to allow all the cells present in the culture plate to firmly adhere on the inside bottom of the plate. Then, the culture supernatant in the plate was removed with the utmost caution not to soak up the cells adhering on the inside bottom of the plate. This means that all the cells present in the cell culture plate exist on the inside bottom of the plate. In this state of things, the dye in the cells was released by the addition of 100 μl of cell lysis solution (isopropanol mixed with 12 M (mol/L) hydrochloric acid) to the culture plate. Absorbance of the cell culture medium was measured at 2 absorbance wavelengths of 570 nm and 690 nm using an absorbance reader (Titertek Multiskan MCC/340, Labsystems, Flow Laboratories Inc., USA). As a result, each polyamine concentration and the total amount of live cells of the cultured peripheral blood monocytes can be confirmed.

Results

The selective inhibition effect on the average fluorescence intensity of CD11a and CD18 by spermine and spermidine was not an apparent effect and was an effect of inhibiting the adhesive function, which is important function of the adhesion molecule LFA-1 composed of CD11a and CD18.

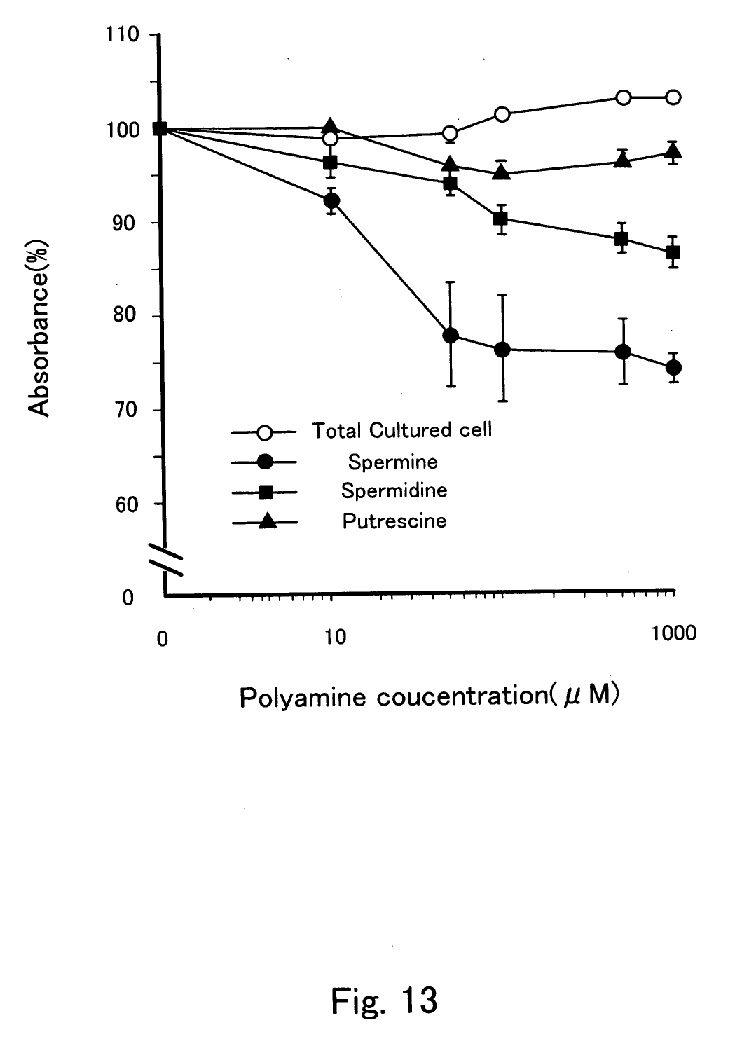

As shown in FIG. 13, when comparison of the number of the cells adhering on the cell culture plate surface was made on the peripheral blood monocytes cultured with varying concentrations of polyamines for 70 to 80 hours, the number of the cells adhering on the plate surface was decreased with increase of spermine and spermidine concentrations. However, the adhesive function of the cells cultured with putrescine was not inhibited even in the culture with the high concentration of putrescine.

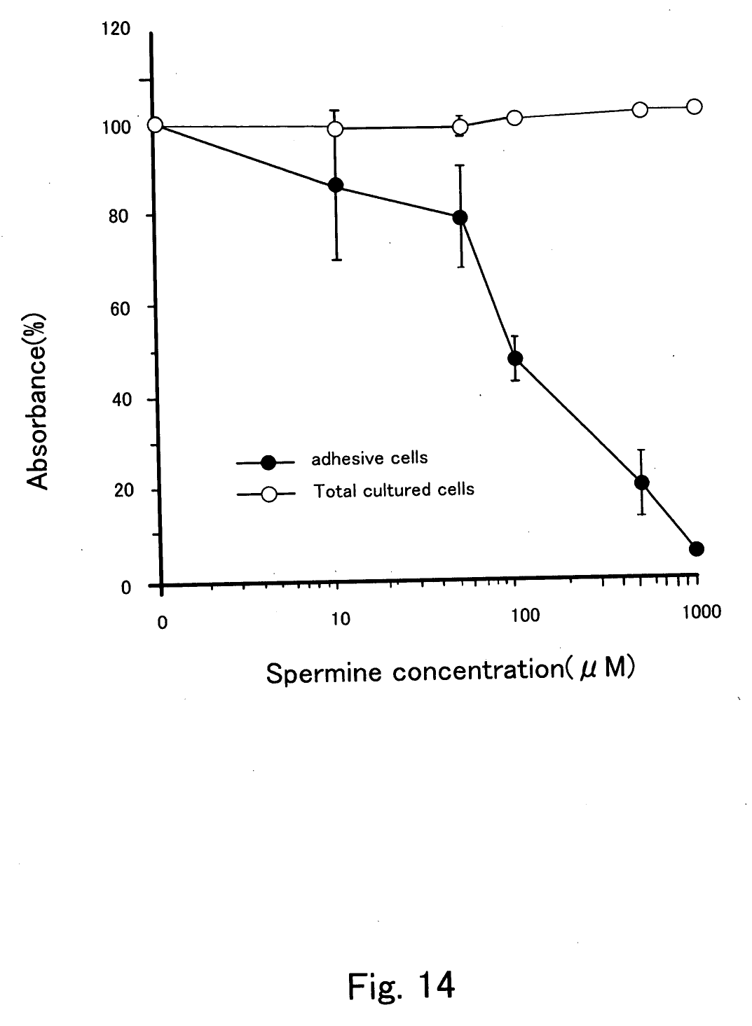

A more remarkable result of the inhibition of cell adhesion to the inside bottom of the culture plate by spermine was obtained by adding, to the cell plate, upward centrifugal force from the bottom thereof and increasing exfoliation of the cells from the cell plate (FIG. 14).

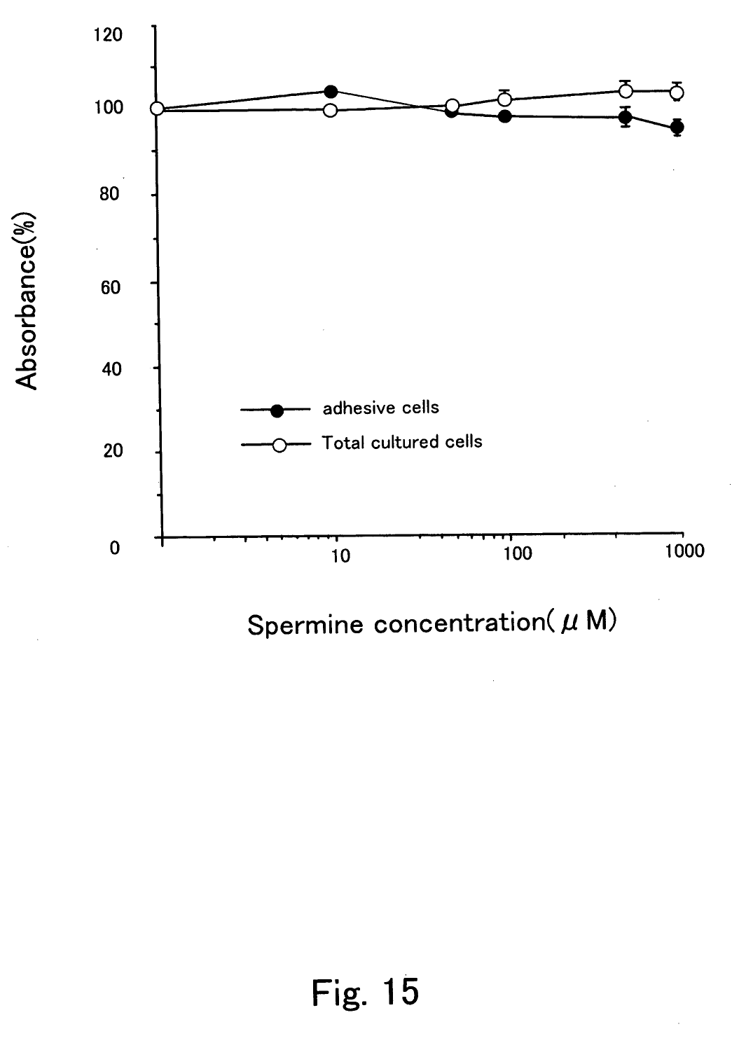

The inhibition effect on cell adhesion to the cell culture plate by spermine and spermidine was not exhibited in the culture for approximately 16 to 24 hours (FIG. 15). This demonstrated that a certain time is required for exhibiting inhibition of LFA-1 function. This result is consistent with the result of Example 2 (Detection of cell membrane differentiation antigen of human peripheral blood monocyte). Namely, in the result of Example 2, it was clarified that the inhibition of average fluorescence intensity of CD11a and CD18 by spermine and spermidine was not exhibited in the culture for a time as short as approximately 24 hours. In this Example 3, it was clarified that the inhibition of function of the LFA-1 molecule composed of CD11a and CD18, that is, the inhibition of cell adhesion to the culture plate also requires time as much as approximately 70 hours when the inhibition of expression of CD11a and CD18 was remarkable in Example 2.

When the MTT dye was taken up into the peripheral blood monocytes in the cell culture plate, followed by measurement of the total number of the cells in the plate, the amount of the dye taken up into the cells had no change and was constant, regardless of spermine concentration (total cultured cells in FIGS. 13, 14, and 15). MTT is taken up only into live cells and reflects cell activity. Thus, no reduction in uptake of the MTT dye and discoloring of the dye in the peripheral blood monocytes cultured with high concentrations of spermine or spermidine also indicates that the cell activity and the number of the cells are not decreased. From this result, it is evident that spermine and spermidine in high concentrations (up to 1 mM) produce no cellular cytotoxicity to the peripheral blood monocytes. Because only the adhesive function of the cells was inhibited by spermine and spermidine, it is evident that the adhesion to the cell culture plate was suppressed.

### Example 4

### Inhibition Effect on Peripheral Blood Monocyte Adhesion to Vascular Endothelial Cell by Polyamines

LFA-1 molecules on the surface of peripheral blood monocytes bind to their ligand CD54 (ICAM-1) in vascular endothelial cells in the early stage of inflammation and in arteriosclerosis, and immunocytes are thereby stimulated. Monocyte and the like secrete various factors involved in inflammation by this stimulation, and inflammation gradually progresses. This mechanism occurs in the initial stage of inflammation and is an important reaction.

Thus, in this Example, study was made on whether or not polyamines inhibit this initial reaction, that is, peripheral blood monocyte adhesion to vascular endothelial cells. Namely, peripheral blood monocytes were cultured for 3 days in cell culture medium containing 0 μM, 100 μM, or 500 μM spermine, spermidine, or putrescine.

In this experiment as well, for removing the influence on the cells of extracellular polyamines present in the culture medium and kept in contact with the cells for a long time, the peripheral blood monocytes were cultured with spermine or spermidine for approximately 16 to 24 hours, then cells were recovered and washed three times with PBS (−) to remove the extracellular polyamines, followed by culture for 48 hours in polyamine-free RPMI1640 culture medium containing 10% human serum.

Vascular endothelial cells used were collected from the vein in umbilical cord from volunteers and subcultured on a culture plate. Techniques of collection of the vascular endothelial cells and methods for the preservation and subculture thereof have no direct bearing on the techniques of the present invention, and the description thereof is therefore omitted.

The endothelial cells of human umbilical cord were cultured in another cell culture plate and spread all over the culture plate (RPMI1640+10% fetal bovine serum was used in culture medium for this cell culture). As a result, the internal environment of blood vessel could be reconstituted on the culture plate. In other words, the adhesion between peripheral blood monocytes and vascular endothelial cells, which actually occurs in human or animal blood vessel can be observed by adding the peripheral blood monocytes to the culture plate having the inside bottom fully covered with the vascular endothelial cells.

In this experiment as well, peripheral blood monocytes which were cultured for 70 to 80 hours in cell culture medium (RPMI1640 supplemented with 10% human serum, 0.1% L-glutamine, and 0.01% penicillin-streptomycin) mixed with the polyamines, or peripheral blood monocytes which after cultured in the cell culture medium containing the polyamines for 16 to 24 hours, were washed with PBS (−) to remove the polyamines present in the cell culture medium, followed by culture in cell culture medium without polyamines for 48 to 56 hours were used.

The cultured peripheral blood monocytes were recovered from the culture plate and washed three times with PBS (−) solution, followed by adjustment of the number of the cells to 5×10⁶ cells/ml. The resulting cells were cultured for 1 hour in 5 μM cell culture medium (RPMI1640+10% fetal bovine serum) containing 2′,7′-bis-(2-carboxyethyl)-5-(and-6)-carboxyfluorescein, acetoxymethyl ester (BCECF-AM) (Molecular Probes, Oregon, USA) (fluorescent reagent).

Next, the peripheral blood monocytes fluorescently labeled with BCECF-AM were respectively adjusted to 1×10⁷ cells/ml, and 100-μL aliquot each thereof was mixed into the cell culture plate in which the vascular endothelial cells were cultured. Following additional culture at 37° C. for 30 minutes, the culture plate was filled with culture medium, then sealed, and inverted at room temperature for 30 minutes. By this procedure, there remain only the peripheral blood monocytes firmly adhering to the vascular endothelial cells covering the inside bottom of the culture plate, whereas nonadhering peripheral blood monocytes can be removed. The culture medium was removed, and the cells were dissolved by addition of 50 μL of aqueous solution of 50 mM Tris-HCl (Wako Pure Chemical Industries LTD, Osaka, Japan) and 0.1% Sodium Dodecyl Sulfate (Sodium Lauryl Sulfate) (SDS) (Wako Pure Chemical Industries LTD, Osaka, Japan) to the culture plate. Fluorescence intensity in this lysate was measured using excitation wavelength of 485 nm and emission wavelength of 583 nm (Fluoroskan, Ascent CE, Labsystems, USA) (Dainippon Pharmaceutical Co., LTD, Tokyo, Japan).

The numbers of the BCECF-AM-incorporating cells cultured under the respective conditions was measured, and fluorescence intensity per cell was measured. The actual numbers of the peripheral blood monocytes adhering to the endothelial cells were respectively calculated from the fluorescence intensity obtained in the experiment described above.

Results

In mechanisms of inflammation and arteriosclerosis onset, peripheral blood monocyte adhesion to vascular endothelial cells in the early stage of the onset is important. When peripheral blood monocytes adhere to vascular endothelial cells, firm cell-cell adhesion is accomplished by binding of LFA-1 existing in the peripheral blood monocytes to ICAM-1 existing in the vascular endothelial cells, and signals of activation are sent into the cells.

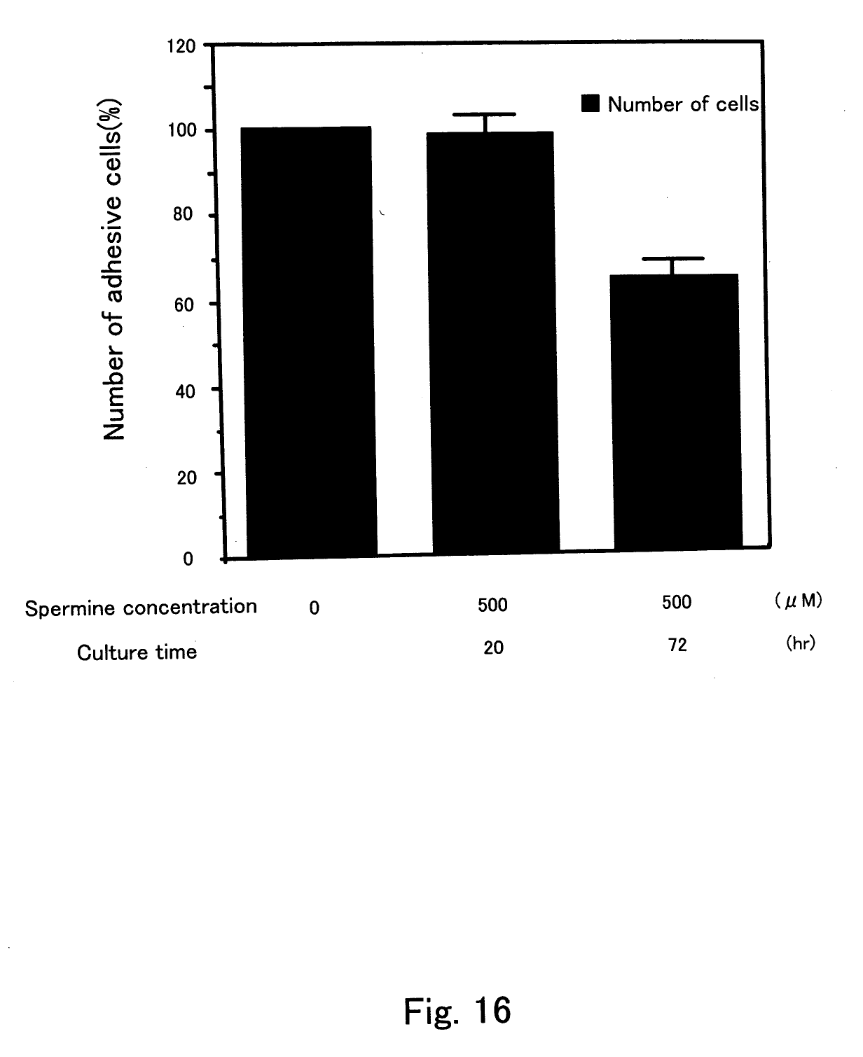

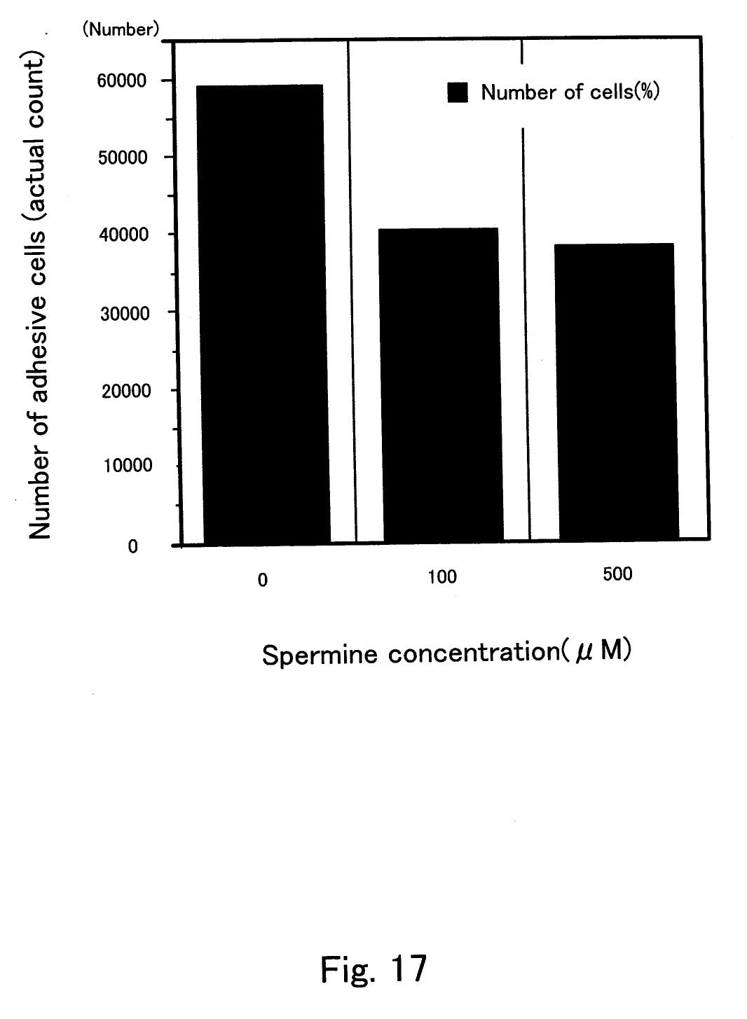

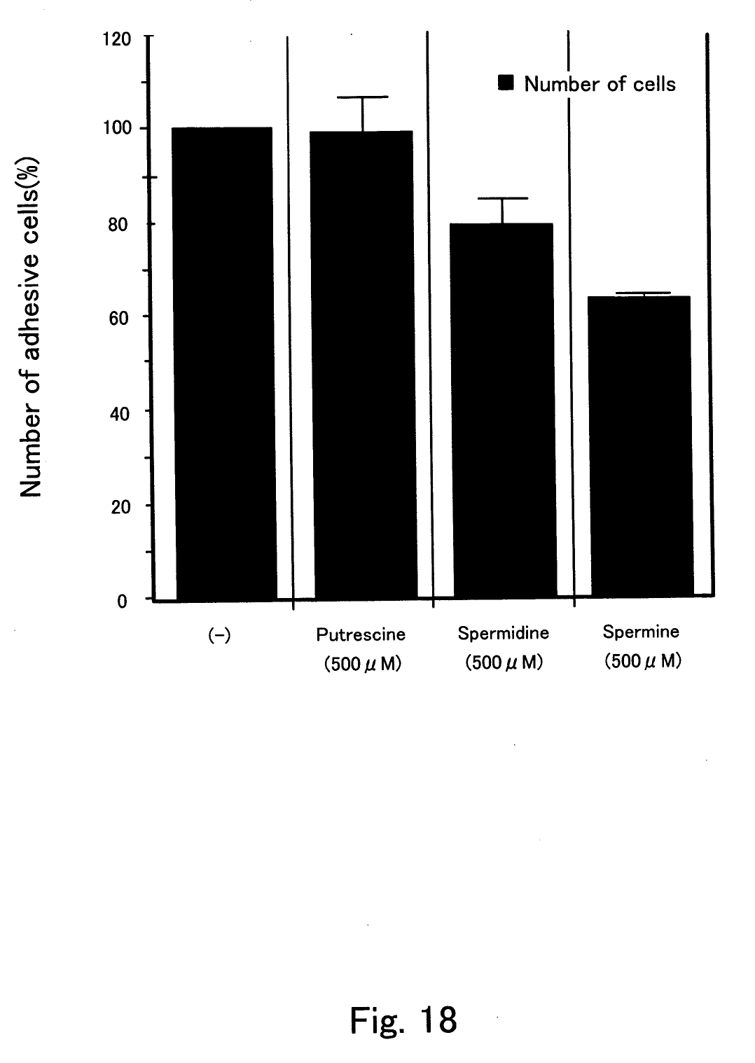

As shown in FIGS. 16 and 17, the number of the cells adhering to the vascular endothelial cells was low in the peripheral blood monocytes cultured with 100 or 500 μM spermine for 72 to 80 hours, as compared with that in the peripheral blood monocytes cultured without spermine. However, as shown in FIG. 16, the number of the adhering cells was not decreased in the culture with 500 μM spermine for 20 hours. As shown in FIG. 18, a similar result was obtained for the culture with spermidine. However, the adhesion to the vascular endothelial cells of the peripheral blood monocytes cultured with putrescine was not inhibited. This demonstrated that spermine and spermidine inhibit the peripheral blood monocyte adhesion to the vascular endothelial cells.

A similar experimental result could also be obtained by using the peripheral blood monocytes cultured with the polyamines for approximately 16 to 24 hours and then cultured in the culture medium without polyamines (FIG. 17). From this result, it is evident that the extracellular spermine or spermidine in high concentrations does not directly inhibit LFA-1 of the peripheral blood monocytes. Namely, as in the results of Examples 2 and 3, it is deduced that spermine and spermidine when taken up into the cells, exert some influence in the cells and inhibit LFA-1 function.

Considering in the light of the results of Examples 2 and 3, the result of this Example is consistent with decrease of average fluorescence intensity of CD11a and CD18 by the polyamines. This could prove decrease of LFA-1 function by spermine and spermidine. This experiment faithfully reproduces the initial step of in-vivo inflammation and arteriosclerosis onset on the culture plate and indicates that spermine and spermidine can attain sufficient effect of preventing inflammation and arteriosclerosis and sufficient improvement of the symptoms when used as anti-inflammation and anti-arteriosclerosis agents.

### Example 5

### Inhibition of Antitumor Activity of Peripheral Blood Monocyte by Polyamines

Peripheral blood monocytes, particularly T-cell lymphocyte, monocyte, macrophage and the like have antitumor activity to tumor cells and can kill the tumor cells by this activity. Since T-cell lymphocyte, monocyte, and macrophage are also contained in peripheral blood monocytes, the peripheral blood monocytes have cellular cytotoxicity to tumor cells. It has been known that when lymphokine (interleukin-2), one of proteins called cytokine which is secreted in trace amounts by cells is cultured with the peripheral blood monocytes, the cellular cytotoxicity is increased. Meanwhile, it is known that LFA-1 function is important for the effect of the peripheral blood monocyte. Accordingly, for the purpose of confirming that the function via LFA-1 of peripheral blood monocytes cultured with polyamines is inhibited, the peripheral blood monocytes cultured with the polyamines were stimulated with interleukin-2 and examined for their antitumor activity.

For peripheral blood monocytes, RPMI1640 mixed with 10% human serum was used, to which spermine was added in the final concentration of 0 μM or 100 μM in the culture medium. The peripheral blood monocytes were cultured in the culture medium containing spermine for 12 to 18 hours and then all recovered from the culture medium. The recovered peripheral blood monocytes were washed three times to remove the culture medium, spermine, and spermidine adhering on the cell surface. The washed cultured peripheral blood monocytes were supplemented with a trace amount (final concentration: 25 U/mL) of a protein which is one kind of cytokine called interleukin-2 (Upstage Biotechnology Inc., Waltham, USA), and cultured in cell culture medium (RPMI1640 containing 10% fetal bovine serum) for 72 hours. Daudi cells (Burkitt lymphoma cells) (Dainippon Pharmaceutical Co., Ltd., Division of Laboratory products, Osaka, Japan) were labeled with radioisotope (⁵¹Cr (sodium dichromate); Daiichi Pure Chemicals Co., Ltd) by addition of the radioisotope to the cell and culture at 37° C. for 1 hour. Subsequently, the peripheral blood monocytes cultured with interleukin-2 as well as the Daudi cells were cultured together in the same cell culture plate. Following culture for 3.5 hours, the culture supernatant was recovered, and the amount of ⁵¹Cr in the culture supernatant was measured with a scintillation counter (γ-counter, LKB). In this experiment, if the tumor cells were destroyed by the peripheral blood monocytes, ⁵¹C in the tumor cells is released into the culture supernatant, and the amount of ⁵¹Cr in the culture supernatant is increased.

Results

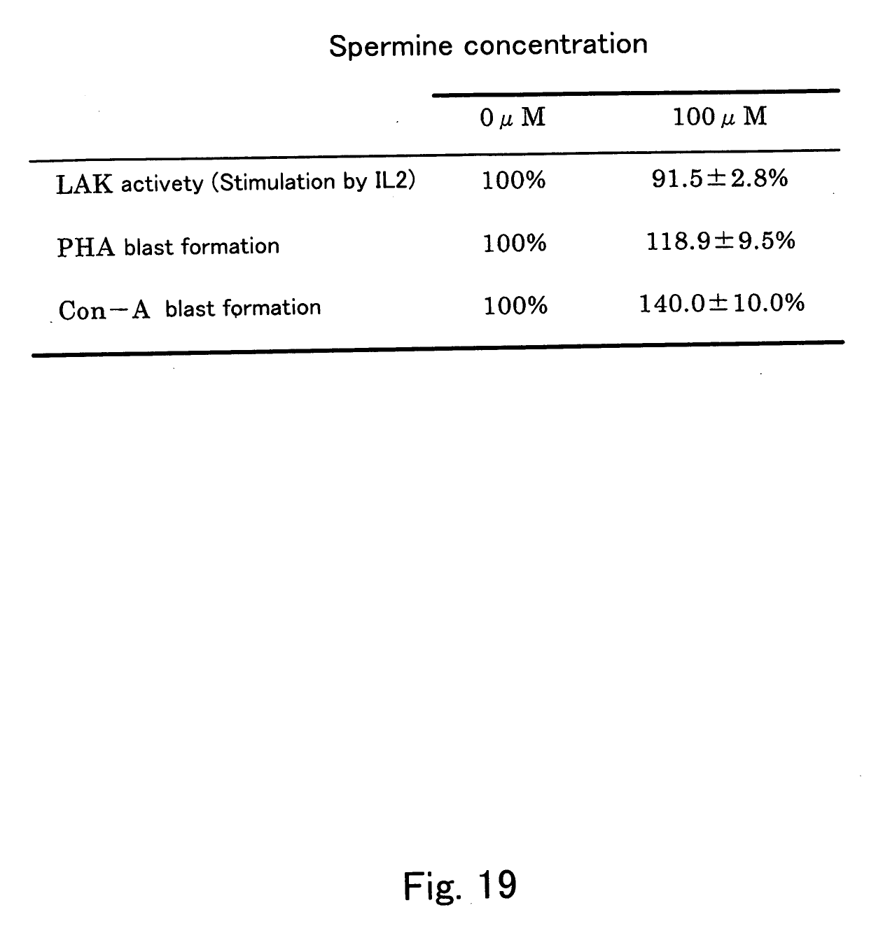

As shown in FIG. 19, the cellular cytotoxicity of the peripheral blood monocytes cultured overnight with 100 μM spermine was reduced. It has been clarified that expression of LFA-1 is most important for exerting cellular cytotoxicity of killer cells in peripheral blood monocytes activated by interleukin-2 (IL-2) (104). It was shown that the LAK activity (cellular cytotoxicity of the cells stimulated with IL-2) of the cells cultured with spermine for 12 to 16 hours is decreased. From this result, the above-described inhibition of LFA-1 function of the peripheral blood monocytes by the polyamines could be recognized with more reliability.

### Example 6

### Examination for Function of Peripheral Blood Monocyte with Use of Polyamines

T-cell lymphocyte contained in peripheral blood monocytes is stimulated on contact with a substance called plant protein lectin (Phytohemagglutinin (hereinafter, PHA) or Concanavalin Agglutinin (Hereinafter, Con-A)), and causes blast transformation. This examination serves as an index capable of quantitatively investigating function of lymphocyte usually contained in peripheral blood monocytes, and this index is decreased in lymphocyte dysfunction such as immunodeficiency.

Peripheral blood monocytes were cultured in cell culture medium (RPMI1640 containing 10% human serum) containing 0 μM or 100 μM spermine for 12 to 18 hours. Following the culture, the cells were washed three times with PBS (−) to remove extracellular polyamines. The washed peripheral blood monocytes were cultured for 64 hours in culture medium (RPMI1640 containing 10% fetal bovine serum) mixed with PHA (Difco Laboratories, Detroit, Mich., USA) or Con-A (Sigma chemical co., St. Louis, USA). Then, ³H-thymidine (Amersham) was added to the cells, followed by additional culture for 8 hours. Following the culture, the peripheral blood monocytes were recovered, and radioactivity of the cells was measured (liquid scintillation counter, LKB-1205, LKB). Since the peripheral blood monocytes activated by mitogen (PHA, Con-A, or the like) cause blast transformation, the cells take up ³H-thymidine therein. Accordingly, function of cell activation can be measured by measuring the amount of intracellular ³H-thymidine.

Results

Interestingly, the blastogenesis by Con-A or PHA of the peripheral blood monocytes cultured with 100 μM spermine or spermidine for approximately 12 to 18 hours was rather enhanced (FIG. 19). In this experiment, the peripheral blood monocytes are allowed to take up the polyamines into their cells by culturing them with spermine or spermidine for a short time up to approximately 18 hours, and then stimulated with Con-A or PHA. Namely, in this experiment as well, it is evident that the presence of not the extracellular polyamines in high concentrations but the intracellular polyamines in high concentrations produces change in function of the peripheral blood monocytes. Furthermore, this examination quantitatively measures general cell function. From the fact that the blastogenesis of the peripheral blood monocytes cultured with spermine or spermidine is enhanced, it is evident that the overall cell functions are activated by spermine, and spermidine. Moreover, this fact is consistent with the fact obtained in the result of Example 2 that decrease of average fluorescence intensity of CD11a and CD18 by spermine and spermidine was selective and that the expression rates of CD11a and CD18 as well as the expression rates and average fluorescence intensity of many other cell membrane differentiation antigens were increased. Accordingly, it was clarified that the inhibition of LFA-1 function by spermine and spermidine is quite selective, and these polyamines rather have effect of enhancing the cell function.

### Example 7

### Cellular Uptake of Polyamines in Culture Medium by Cultured Peripheral Blood Monocyte

It was considered that a certain time is required for exhibiting the inhibition effect of spermine and spermidine on LFA-1, and spermine and spermidine change intracellular signals after taken up into the cells.

This experiment investigated cellular uptake of polyamines in culture medium by cultured peripheral blood monocytes. The peripheral blood monocytes were cultured in culture medium supplemented with 500 μM putrescine, spermidine, or spermine. Following culture for 16 hours, the cells were recovered from the culture plate and placed into a 50-mL tube. The tube was centrifuged (4° C., 10 min., 100 rpm), and the whole culture medium was aspirated. The cells were washed with 50 mL PBS (−) and centrifuged again, and the supernatant was aspirated. This procedure was repeated three times, and PBS (−) was added to the cells to adjust the cell concentration to approximately 1×10⁷ cells/mL, followed by freezing to −20° C. As a result of this single procedure, up to approximately 100 μL culture medium or cell suspension remains in the tube after the centrifugation. Provided that 500 μL thereof remains, then dilution gets 100-fold by suspending the cells again with 50 mL PBS. When the same procedure is repeated, the polyamines originally contained in the cell culture medium are diluted 1000000-fold by simple arithmetic. The original concentration (500 μM) of the polyamines in the cell culture medium ends in a concentration up to 500 μM by repeating the procedure three times. The frozen cell suspension was subjected to high-performance liquid chromatography (LCMS-2010, Shimadzu Corp., Kyoto, Japan) to measure polyamine concentration in the cell suspension.

Results

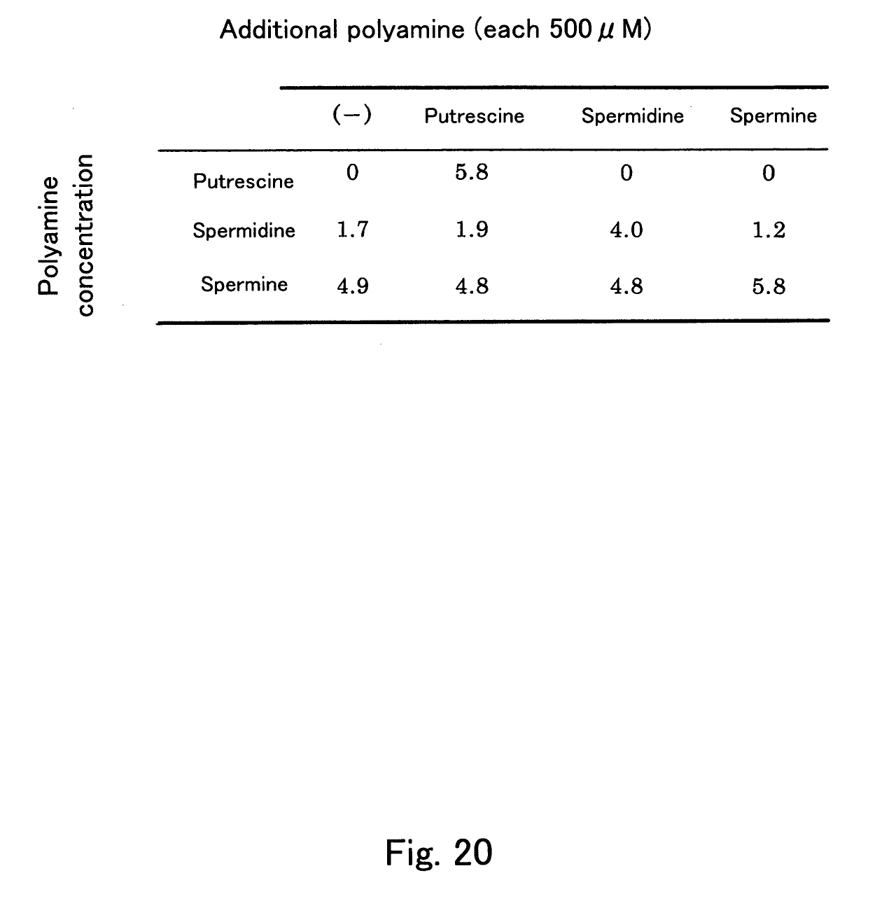

FIG. 20 shows polyamine concentration per 1×10⁷ cells/mL of the cell suspension obtained by suspending the peripheral blood monocytes cultured with in the culture medium supplemented with 500 μM each of the polyamines for 16 hours. Only the concentration of the cultured polyamine was increased in the peripheral blood monocytes cultured with each polyamine. As described above, the unit of the extracellular polyamines was supposed to be pM, whereas the unit of the measured polyamine concentration of the cell suspension was μM. Accordingly, it is considered that the polyamines contained in the culture medium exert little influence on concentration measurement. Since the measured cell suspension was the one in which the cells were disrupted by freezing to leak the intracellular polyamines into the suspension, it is considered that the measurement value reflects polyamine concentration in the cells. Thus, it is obvious that the polyamines in the culture medium are taken up into the peripheral blood monocytes.

As described above, the present inventor found that spermine and spermidine contained in cells inhibit the expression of CD11a and CD18 on the surface of peripheral blood monocytes (lymphocyte, monocyte, and macrophage) which are immunocytes in human blood and inhibit the function of the cell surface molecule LFA-1 composed of these two molecules. This inhibition is selective and is not exerted on other cell membrane antigen molecules, most of which are rather promoted. The general index of cell function is also enhanced.

For increasing spermine and spermidine concentrations in the cells, it is only necessary to orally or parenterally ingest spermine and spermidine. Therefore, the present invention can be practiced easily and provides quite useful selective inhibitors of LFA-1.

Furthermore, it has already been clarified that effects of inhibiting arteriosclerosis onset, rejection of transplanted organs, and symptoms of one of autoimmune diseases (psoriasis) are obtained by inhibiting LFA-1 function in human. Thus, the inhibitors of the present invention can be expected to have sufficient effects as agents for the treatment or inhibition of these diseases.

It has also been clarified that LFA-1 plays an important role in the pathology of each disease of autoimmune disease (type I diabetes (insulin-dependent diabetes mellitus), Graves' disease (Basedow disease), Hashimoto disease, autoimmune arthritis (Lyme arthritis, chronic rheumatoid arthritis), autoimmune cerebrospinal peripheral neuritis or degeneration, Sjogren's syndrome, uveitis, retinitis or degeneration, autoimmune renal disease (glomerulonephritis and the like), inflammatory bowel disease (Crohn disease, ulcerative colitis and the like), and primary cholangitis), allergic disease, ischemic reperfusion injury, and diabetic retinopathy. Moreover, when LFA-1 function of animals with diseases similar to these human diseases is inhibited by administering anti-LFA-1 antibody to the animals, inhibition of progression of the disease and reduction of the symptoms can be conducted. From these points of view and from the listed findings about the relationship of CD11a and LFA-1 with the diseases and their related documents as described in “Best Mode for Carrying Out the Invention,” it is evident that the inhibitors of the present invention are capable of preventing the diseases and improving the symptoms.

It was clarified that even if peripheral blood monocytes are cultured in cell culture medium containing spermidine or spermine, expression of CD11a and CD18 is not immediately inhibited, and a certain time (usually approximately 72 hours, at least 24 hours or more) is required for the inhibition. This indicates that when spermine and spermidine inhibit expression of CD11a and CD18 on the cell surface, the spermine molecule, spermidine molecule, or their related molecule does not act directly on CD11a and CD18. Considering that spermine and spermidine are easily taken up into the cells, it is considered that change of intracellular spermine and spermidine concentrations changes intracellular signal (information) transduction, and expression intensity of CD11a and CD18 on the cell surface is thereby inhibited. This can be deduced from the inhibited expression intensity of CD11a and CD18 in the peripheral blood monocytes which were cultured spermine for only 16 to 24 hours and then cultured in culture medium without spermine for 48 to 56 hours. Thus, it is considered that when intracellular spermine or spermidine concentration is increased, some change of information is added to the intracellular signal transduction system promoting expression of CD11a and CD18, with the result that the expression of CD11a and CD18 on the cell surface is inhibited.

From previous studies, it has been thought that an amount of the polyamines ingested in a day in adult is about 350 to 550 μmol from an amount of the polyamines contained in foods (105) and average meal in a day (106).

It has been reported in large numbers that when spermine and spermidine are administered in high concentration, intestinal mucosa is damaged. However, it has also been clarified that when spermine and spermidine are administered in suitable concentration (not over about 0.1%), they have effects of facilitating the growth of the intestinal mucosa (107).

Moreover, it has been reported that spermine has toxicity when contained at 0.2% or more in meal but produces good effect when contained in concentration that is an order of magnitude smaller, and orally administered spermidine produces good effect at 0.05% (108).

In many experiments using animals, an amount of spermine and spermidine exerting acute toxicity has already been clarified (109).

Til et al. have reported that in the acute toxicity test using rats, 50% lethal dose (LD50) of spermidine and spermine is 600 mg/kg of body weight. They have also reported that in the subacute toxicity test (administration for 6 weeks), no side effect is observed in spermidine administered in an amount up to 83 mg/kg of body weight/day and spermine administered in an amount up to 19 mg/kg of body weight/day. Furthermore, the scope of the study has clarified not only the dose expressing acute toxicity but also the concentration of the substances taken internally as described above. Moreover, both spermine and spermidine have water absorbency and are stable in the form of aqueous solution. As described above, it has been clarified not only that these substances are absorbed in their original forms from intestine and carried to each tissue in the body but also that polyamine concentration in peripheral blood monocyte is also increased.

In the studies conducted by the present inventor, increase of spermine or spermidine concentration from 100 to 500 μM in culture medium was sufficient for inhibiting LFA-1 of peripheral blood monocyte. Water makes up 60% of body weight of a human adult, of which the water content intercellularly present and the extracellular fluid extracellularly present are 40% and 20%, respectively. Given that a human body weight is 50 kg, the amount of the extracellular fluid is calculated at 10 kg (approximately 10 L). Thus, administration of spermine or spermidine in a concentration of 500 μmol in this extracellular fluid requires 5 mmol spermine or spermidine from the calculation of 500 μmol (μmol/L)×10 L=5000 μmol=5 mmol. Because the molecular weights of spermine and spermidine are 202.34 and 145.24, respectively, 5 mmol spermine and spermidine correspond to approximately 1,012 mg and 726 mg, respectively. This corresponds to 20.23 mg of spermine/kg of body weight and 14.5 mg of spermidine/kg of body weight, and both spermidine and spermine provided in these amounts are said to be safe. These figures are those for theoretically exerting sufficient inhibition effect on LFA-1 in single administration. However, spermine and spermidine gradually exert inhibition effect on LFA-1 after taken up into cells. Therefore, consecutive administration of them in small amounts is more realistic.

According to the report about effect of spermine and spermidine concentration in meal on intestinal mucosa, it has been clarified that the amount of approximately 0.1 to 0.05% spermine and spermidine in foods does not damage intestinal mucosa and has good effects such as promotion of mucosa proliferation. Therefore, practically, a method by which an aqueous solution having the spermine or spermidine concentration of approximately 0.02 to 0.04% is taken internally is most recommended in consideration of safety. Moreover, gradual instillation administration of an agent for instillation such as saline mixed with a similar concentration of spermine or spermidine is considered possible.

Thus, the highest possible amount administered by internal administration or intravenous administration through instillation of a solution of 500 mL/day of the polyamine is 200 mg from the calculation of 500 mL (=g)×0.04%=200 mg. This amount corresponds to approximately 988 μmol spermine and 1,377 μmol spermidine. In consideration of the amount (350 to 550 μmol) expected to be ingested by an adult from meal, those amounts are sufficiently reasonable from a safety standpoint. The amounts are considered to be amounts capable of exerting sufficient treatment effects. In administration for the inhibition of the diseases, smaller amounts of the polyamines are preferably administered. In a method for administering spermine and spermidine, the upper limit per kg of body weight at one dose or daily dose is given at up to 200 μmol in an aqueous or alcohol solution having any one or both of spermine and spermidine concentrations ranging from 0.1% to 0.001%.

In Examples, spermine and spermidine among polyamines were mainly used, because they are typical biogenic polyamines. It will be evident that other polyamines have similar effects.

### REFERENCE

1. Yusuf-Makagiansar H, Anderson M E, Yakovleva T V, Murray J S, Siahaan T J.:Inhibition of LFA-1/ICAM-1 and VLA-4/VCAM-1 as a therapeutic approach to inflammation and autoimmune diseases.:Med Res Rev. 2002, 22(2):146-67.
2. Staunton D E, Dustin M L, Springer T A.:Functional cloning of ICAM-2, a cell adhesion ligand for LFA-1 homologous to ICAM-1.:Nature. 1989, 339(6219):61-4.
3. Dedrick R L, Walicke P, Garovoy M.:Anti-adhesion antibodies efalizumab, a humanized anti-CD11a monoclonal antibody.:Transpl Immunol. 2002, 9(2-4):181-6.
4. Guerette B, Moisset P A, Huard C, Tardif F, Gravel C, Tremblay J P.:Inflammatory damage following first-generation replication-defective adenovirus controlled by anti-LFA-1.:J Leukoc Biol. 1997, 61(4):533-8.
5. Li X, Abdi K, Rawn J, Mackay C R, Mentzer S J.:LFA-1 and L-selectin regulation of recirculating lymphocyte tethering and rolling on lung microvascular endothelium.:Am J Respir Cell Mol. Biol. 1996, 14(4):398-406.
6. Shang X Z, Issekutz A C.:Enhancement of monocyte transendothelial migration by granulocyte-macrophage colony-stimulating factor: requirement for chemoattractant and CD11a/CD18 mechanisms.:Eur J. Immunol. 1999, 29(11):3571-82.
7. Mentzer S J, Crimmins M A, Herrmann S H.:Functional domains of the CD11a adhesion molecule in lymphokine activated killer (LAK)-mediated cytolysis.:J Clin Lab Immunol. 1988, 27(4):155-61.
8. Werther W A, Gonzalez T N, O'Connor S J, McCabe S, Chan B, Hotaling T, Champe M, Fox J A, Jardieu P M, Berman P W, Presta L G.:Humanization of an anti-lymphocyte function-associated antigen (LFA)-1 monoclonal antibody and reengineering of the humanized antibody for binding to rhesus LFA-1.:J. Immunol. 1996, 157(11):4986-95.
9. Dedrick R L, Walicke P, Garovoy M.:Anti-adhesion antibodies efalizumab, a humanized anti-CD11a monoclonal antibody.:Transpl Immunol. 2002, 9(2-4):181-6.
10. Papp K, Bissonnette R, Krueger J G, Carey W, Gratton D, Gulliver W P, Lui H, Lynde C W, Magee A, Minier D, Ouellet J P, Patel P, Shapiro J, Shear N H, Kramer S, Walicke P, Bauer R, Dedrick R L, Kim S S, White M, Garovoy M R.:The treatment of moderate to severe psoriasis with a new anti-CD11a monoclonal antibody.:J Am Acad Dermatol. 2001, 45(5):665-74.
11. Gottlieb A B, Krueger J G, Wittkowski K, Dedrick R, Walicke P A, Garovoy M.:Psoriasis as a model for T-cell-mediated disease: immunobiologic and clinical effects of treatment with multiple doses of efalizumab, an anti-CD11a antibody.:Arch Dermatol. 2002, 138(5):591-600.
12. Dedrick R L, Walicke P, Garovoy M.:Anti-adhesion antibodies efalizumab, a humanized anti-CD11a monoclonal antibody.:Transpl Immunol. 2002, 9(2-4):181-6.
13. Gadek T R, Burdick D J, McDowell R S, Stanley M S, Marsters J C Jr, Paris K J, Oare D A, Reynolds M E, Ladner C, Zioncheck K A, Lee W P, Gribling P, Dennis M S, Skelton N J, Tumas D B, Clark K R, Keating S M, Beresini M H, Tilley J W, Presta L G, Bodary S C.:Generation of an LFA-1 antagonist by the transfer of the ICAM-1 immunoregulatory epitope to a small molecule.:Science. 2002, 295(5557):1086-9.
14. Kallen J, Welzenbach K, Ramage P, Geyl D, Kriwacki R, Legge G, Cottens S, Weitz-Schmidt G, Hommel U.:Structural basis for LFA-1 inhibition upon lovastatin binding to the CD11a I-domain.:J Mol. Biol. 1999, 292(1):1-9.
15. Kobashigawa J A, Katznelson S, Laks H, Johnson J A, Yeatman L, Wang X M, Chia D, Terasaki P I, Sabad A, Cogert G A, et al.:Effect of pravastatin on outcomes after cardiac transplantation.:N Engl J Med. 1995, 333(10):621-7.
16. Weitz-Schmidt G, Welzenbach K, Brinkmann V, Kamata T, Kallen J, Bruns C, Cottens S, Takada Y, Hommel U.:Statins selectively inhibit leukocyte function antigen-1 by binding to a novel regulatory integrin site.:Nat Med. 2001, 7(6):687-92.
17. Yusuf-Makagiansar H, Anderson M E, Yakovleva T V, Murray J S, Siahaan T J.:Inhibition of LFA-1/ICAM-1 and VLA-4/VCAM-1 as a therapeutic approach to inflammation and autoimmune diseases.:Med Res Rev. 2002, 22(2):146-67.
18. Shier P, Otulakowski G, Ngo K, Panakos J, Chourmouzis E, Christjansen L, Lau C Y, Fung-Leung W P.:Impaired immune responses toward alloantigens and tumor cells but normal thymic selection in mice deficient in the beta2 integrin leukocyte function-associated antigen-1.:J. Immunol. 1996, 157(12):5375-86.
19. Garcia N, Mileski W J, Lipsky P.:Differential effects of monoclonal antibody blockade of adhesion molecules on in vivo susceptibility to soft tissue infection.:Infect Immun. 1995, 63(10):3816-9.
20. Scalabrino G, Ferioli M E.:Polyamines in mammalian ageing: an oncological problem, tool A review.:Mech Ageing Dev. 1984, 26(2-3):149-64.
21. Casti A, Orlandini G, Reali N, Bacciottini F, Vanelli M, Bernasconi S.:Pattern of blood polyamines in healthy subjects from infancy to the adult age.:J Endocrinol Invest. 1982, 5(4):263-6.
22. Das R, Kanungo M S.:Activity and modulation of ornithine decarboxylase and concentrations of polyamines in various tissues of rats as a function of age.:Exp Gerontol. 1982, 17(2):95-103.
23. Bardocz S, Brown D S, Grant G, Pusztai A.:Luminal and basolateral polyamine uptake by rat small intestine stimulated to grow by Phaseolus vulgaris lectin phytohaemagglutinin in vivo.:Biochim Biophys Acta. 1990, 1034(1):46-52.
24. Cohen L F, Lundgren D W, Farrell P M.:Distribution of spermidine and spermine in blood from cystic fibrosis patients and control subjects.:Blood. 1976, 48(3):469-75.
25. Nishiguchi S, Tamori A, Koh N, Fujimoto S, Takeda T, Shiomi S, Oka H, Yano Y, Otani S, Kuroki T.:Erythrocyte-binding polyamine as a tumor growth marker for human hepatocellular carcinoma.:Hepatogastroenterology. 2002, 49(44):504-7.
26. Scalabrino G, Ferioli M E.:Polyamines in mammalian ageing: an oncological problem, tool A review.:Mech Ageing Dev. 1984, 26(2-3):149-64.
27. Casti A, Orlandini G, Reali N, Bacciottini F, Vanelli M, Bernasconi S.:Pattern of blood polyamines in healthy subjects from infancy to the adult age.:J Endocrinol Invest. 1982, 5(4):263-6.
28. Cooper K D, Shukla J B, Rennert O M.:Polyamine distribution in cellular compartments of blood and in aging erythrocytes.:Clin Chim Acta. 1976, 73(1):71-88.
29. Nishimura K, Araki N, Ohnishi Y, Kozaki S.:Effects of dietary polyamine deficiency on tripanosoma gambiense infection in rats.:Experimental Parasitology 2001, 97; 95-101
30. Bardócz S, Grant G, Brown D S, Ralph A, and Pusztai A.:Polyamines in food—implications for growth and health.:J. Nutr. Biochem. 1993, 4:66-71.
31. Kobashigawa J A, Katznelson S, Laks H, Johnson J A, Yeatman L, Wang X M, Chia D, Terasaki P I, Sabad A, Cogert G A, et al.:Effect of pravastatin on outcomes after cardiac transplantation.:N Engl J. Med. 1995, 333(10):621-7.
32. Weitz-Schmidt G, Welzenbach K, Brinkmann V, Kamata T, Kallen J, Bruns C, Cottens S, Takada Y, Hommel U.:Statins selectively inhibit leukocyte function antigen-1 by binding to a novel regulatory integrin site.:Nat Med. 2001, 7(6):687-92.
33. Kallen J, Welzenbach K, Ramage P, Geyl D, Kriwacki R, Legge G, Cottens S, Weitz-Schmidt G, Hommel U.:Structural basis for LFA-1 inhibition upon lovastatin binding to the CD11a I-domain.:J Mol. Biol. 1999, 292(1):1-9.
34. Kawakami A, Tanaka A, Nakajima K, Shimokado K, Yoshida M.:Atorvastatin attenuates remnant lipoprotein-induced monocyte adhesion to vascular endothelium under flow conditions.:Circ Res. 2002, 91(3):263-71.
35. Mine S, Tabata T, Wada Y, Fujisaki T, Iida T, Noguchi N, Niki E, Kodama T, Tanaka Y.:Oxidized low density lipoprotein-induced LFA-1-dependent adhesion and transendothelial migration of monocytes via the protein kinase C pathway.:Atherosclerosis. 2002, 160(2):281-8.
36. Nie Q, Fan J, Haraoka S, Shimokama T, Watanabe T.:Inhibition of mononuclear cell recruitment in aortic intima by treatment with anti-ICAM-1 and anti-LFA-1 monoclonal antibodies in hypercholesterolemic rats: implications of the ICAM-1 and LFA-1 pathway in atherogenesis.:Lab Invest. 1997, 77(5):469-82.)
37. Suzuki J, Isobe M, Yamazaki S, Horie S, Okubo Y, Sekiguchi M.:Inhibition of accelerated coronary atherosclerosis with short-term blockade of intercellular adhesion molecule-1 and lymphocyte function-associated antigen-1 in a heterotopic murine model of heart transplantation.:J Heart Lung Transplant. 1997, 16(11):1141-8.
38. Russell P S, Chase C M, Colvin R B.:Coronary atherosclerosis in transplanted mouse hearts. IV Effects of treatment with monoclonal antibodies to intercellular adhesion molecule-1 and leukocyte function-associated antigen-1.:Transplantation. 1995, 60(7):724-9.
39. Papp K, Bissonnette R, Krueger J G, Carey W, Gratton D, Gulliver W P, Lui H, Lynde C W, Magee A, Minier D, Ouellet J P, Patel P, Shapiro J, Shear N H, Kramer S, Walicke P, Bauer R, Dedrick R L, Kim S S, White M, Garovoy M R.:The treatment of moderate to severe psoriasis with a new anti-CD11a monoclonal antibody.:J Am Acad Dermatol. 2001, 45(5):665-74.
40. Gottlieb A B, Krueger J G, Wittkowski K, Dedrick R, Walicke P A, Garovoy M.:Psoriasis as a model for T-cell-mediated disease: immunobiologic and clinical effects of treatment with multiple doses of efalizumab, an anti-CD11a antibody.:Arch Dermatol. 2002, 138(5):591-600.
41. Gottlieb A, Krueger J G, Bright R, Ling M, Lebwohl M, Kang S, Feldman S, Spellman M, Wittkowski K, Ochs H D, Jardieu P, Bauer R, White M, Dedrick R, Garovoy M.:Effects of administration of a single dose of a humanized monoclonal antibody to CD11a on the immunobiology and clinical activity of psoriasis.:J Am Acad Dermatol. 2000, 42(3):428-35.
42. Dedrick R L, Walicke P, Garovoy M.:Anti-adhesion antibodies efalizumab, a humanized anti-CD11a monoclonal antibody.:Transpl Immunol. 2002, 9(2-4):181-6.
43. Zeigler M, Chi Y, Tumas D B, Bodary S, Tang H, Varani J.:Anti-CD11a ameliorates disease in the human psoriatic skin-SCID mouse transplant model: comparison of antibody to CD11a with Cyclosporin A and clobetasol propionate.:Lab Invest. 2001, 81(9):1253-61.
44. Mysliwiec J, Kretowski A, Kinalski M, Kinalska I.:CD11a expression and soluble ICAM-1 levels in peripheral blood in high-risk and overt type 1 diabetes subjects.:Immunol Lett. 1999, 70(1):69-72.
45. Moriyama H, Yokono K, Amano K, Nagata M, Hasegawa Y, Okamoto N, Tsukamoto K, Miki M, Yoneda R, Yagi N, Tominaga Y, Kikutani H, Hioki K, Okumura K, Yagita H, Kasuga M.:Induction of tolerance in murine autoimmune diabetes by transient blockade of leukocyte function-associated antigen-1/intercellular adhesion molecule-1 pathway.:J. Immunol. 1996, 157(8):3737-43.
46. Herold K C, Vezys V, Gage A, Montag A G.:Inhibition of autoimmune diabetes by treatment with anti-LFA-1 and anti-ICAM-1 monoclonal antibodies.:Cell Immunol. 1994, 157(2):489-500.
47. Hasegawa Y, Yokono K, Taki T, Amano K, Tominaga Y, Yoneda R, Yagi N, Maeda S, Yagita H, Okumura K, et al.:Inhibition of autoimmune insulin-dependent diabetes in non-obese diabetic mice by anti-LFA-1 and anti-ICAM-1 mAb.:Int Immunol. 1994, 6(6):831-8.
48. Kretowski A, Mysliwiec J, Kinalska I.:The alterations of CD11A expression on peripheral blood lymphocytes/monocytes and CD62L expression on peripheral blood lymphocytes in Graves' disease and type 1 diabetes.:Rocz Akad Med Bialymst. 1999, 44:151-9.
49. Guerin V, Bene M C, Amiel C, Hartemann P, Leclere J, Faure G.:Decreased lymphocyte function-associated antigen-1 molecule expression on peripheral blood lymphocytes from patients with Graves' disease.:J Clin Endocrinol Metab. 1989, 69(3):648-53.
50. Arao T, Morimoto I, Kakinuma A, Ishida O, Zeki K, Tanaka Y, Ishikawa N, Ito K, Ito K, Eto S.:Thyrocyte proliferation by cellular adhesion to infiltrating lymphocytes through the intercellular adhesion molecule-1/lymphocyte function-associated antigen-1 pathway in Graves' disease.:J Clin Endocrinol Metab. 2000, 85(1):382-9.
51. Bagnasco M, Pesce G P, Caretto A, Paolieri F, Pronzato C, Villaggio B, Giordano C, Betterle C, Canonica G W.:Follicular thyroid cells of autoimmune thyroiditis may coexpress ICAM-1 (CD54) and its natural ligand LFA-1 (CD11a/CD18).:J Allergy Clin Immunol. 1995, 95(5 Pt 1):1036-43
52. Marazuela M, Postigo A A, Acevedo A, Diaz-Gonzalez F, Sanchez-Madrid F, de Landazuri M O.:Adhesion molecules from the LFA-1/ICAM-1,3 and VLA-4/VCAM-1 pathways on T lymphocytes and vascular endothelium in Graves' and Hashimoto's thyroid glands.:Eur J. Immunol. 1994, 24(10):2483-90.)
53. Steere A C, Gross D, Meyer A L, Huber B T.:Autoimmune mechanisms in antibiotic treatment-resistant lyme arthritis.:J Autoimmun. 2001, 16(3):263-8.
54. Gross D M, Forsthuber T, Tary-Lehmann M, Etling C, Ito K, Nagy Z A, Field J A, Steere A C, Huber B T.:Identification of LFA-1 as a candidate autoantigen in treatment-resistant Lyme arthritis.:Science. 1998, 281(5377):703-6.
55. Birner U, Issekutz T B, Walter U, Issekutz A C.:The role of alpha(4) and LFA-1 integrins in selectin-independent monocyte and neutrophil migration to joints of rats with adjuvant arthritis.:Int Immunol. 2000, 12(2):141-50.
56. Gordon E J, Myers K J, Dougherty J P, Rosen H, Ron Y.:Both anti-CD11a (LFA-1) and anti-CD11b (MAC-1) therapy delay the onset and diminish the severity of experimental autoimmune encephalomyelitis.:J Neuroimmunol. 1995, 62(2):153-60.
57. Willenborg D O, Staykova M A, Miyasaka M.:Short term treatment with soluble neuroantigen and anti-CD11a (LFA-1) protects rats against autoimmune encephalomyelitis: treatment abrogates autoimmune disease but not autoimmunity.:J. Immunol. 1996, 157(5):1973-80.
58. Archelos J J, Maurer M, Jung S, Miyasaka M, Tamatani T, Toyka K V, Hartung H P.:Inhibition of experimental autoimmune neuritis by an antibody to the lymphocyte function-associated antigen-1.:Lab Invest. 1994, 70(5):667-75.
59. Inoue A, Koh C S, Yamazaki M, Ichikawa M, Isobe M, Ishihara Y, Yagita H, Kim B S.:Anti-adhesion molecule therapy in Theiler's murine encephalomyelitis virus-induced demyelinating disease.:Int Immunol. 1997 December; 9(12):1837-47.
60. Kapsogeorgou E K, Dimitriou I D, Abu-Helu R F, Moutsopoulos H M, Manoussakis M N.:Activation of epithelial and myoepithelial cells in the salivary glands of patients with Sjogren's syndrome: high expression of intercellular adhesion molecule-1 (ICAM.1) in biopsy specimens and cultured cells.:Clin Exp Immunol. 2001, 124(1):126-33.)
61. Takahashi M, Mimura Y, Hayashi Y.:Role of the ICAM-1/LFA-1 pathway during the development of autoimmune dacryoadenitis in an animal model for Sjogren's syndrome.:Pathobiology. 1996, 64(5):269-74.
62. Hayashi Y, Haneji N, Yanagi K, Higashiyama H, Yagita H, Hamano H.:Inhibition of adoptive transfer of murine Sjogren's syndrome into severe combined immunodeficient (SCID) mice by antibodies against intercellular adhesion molecule-1 (ICAM-1) and lymphocyte function-associated antigen-1 (LFA-1).:Clin Exp Immunol. 1995, 102(2):360-7.
63. Uchio E, Kijima M, Tanaka S, Ohno S.:Suppression of experimental uveitis with monoclonal antibodies to ICAM-1 and LFA-1.:Invest Ophthalmol Vis Sci. 1994, 35(5):2626-31.
64. Whitcup S M, DeBarge L R, Caspi R R, Harning R, Nussenblatt R B, Chan C C.:Monoclonal antibodies against ICAM-1 (CD54) and LFA-1 (CD11a/CD18) inhibit experimental autoimmune uveitis.:Clin Immunol Immunopathol. 1993, 67(2): 143-50.
65. Ando K, Fujino Y, Mochizuki M.:Effects of monoclonal antibodies directed at cell surface molecules on murine experimental autoimmune uveoretinitis.:Graefes Arch Clin Exp Ophthalmol. 1999, 237(10):848-54.
66. Nishikawa K, Guo Y J, Miyasaka M, Tamatani T, Collins A B, Sy M S, McCluskey R T, Andres G.:Antibodies to intercellular adhesion molecule 1/lymphocyte function-associated antigen 1 prevent crescent formation in rat autoimmune glomerulonephritis.:J Exp Med. 1993, 177(3):667-77.
67. Kawasaki K, Yaoita E, Yamamoto T, Tamatani T, Miyasaka M, Kihara I.:Antibodies against intercellular adhesion molecule-1 and lymphocyte function-associated antigen-1 prevent glomerular injury in rat experimental crescentic glomerulonephritis.:J. Immunol. 1993, 150:1074-1083.
68. Kootstra C J, Van Der Giezen D M, Van Krieken J H, De Heer E, Bruijn J A.:Effective treatment of experimental lupus nephritis by combined administration of anti-CD11a and anti-CD54 antibodies.:Clin Exp Immunol. 1997, 108(2):324-32.
69. Taniguchi T, Tsukada H, Nakamura H, Kodama M, Fukuda K, Saito T, Miyasaka M, Seino Y.:Effects of the anti-ICAM-1 monoclonal antibody on dextran sodium sulphate-induced colitis in rats.:J Gastroenterol Hepatol. 1998, 13(9):945-9.
70. Vainer B, Nielsen O H, Horn T.:Comparative studies of the colonic in situ expression of intercellular adhesion molecules (ICAM-1, -2, and -3), beta2 integrins (LFA-1, Mac-1, and p150,95), and PECAM-1 in ulcerative colitis and Crohn's disease.:Am J Surg Pathol. 2000, 24(8):1115-24.
71. Kimura T, Suzuki K, Inada S, Hayashi A, Isobe M, Matsuzaki Y, Tanaka N, Osuga T, Fujiwara M.:Monoclonal antibody against lymphocyte function-associated antigen 1 inhibits the formation of primary biliary cirrhosis-like lesions induced by murine graft-versus-host reaction.:Hepatology. 1996, 24(4):888-94.
72. Shiina M, Kobayashi K, Mano Y, Ueno Y, Ishii M, Shimosegawa T.:Up-regulation of CD11a (LFA-1) expression on peripheral CD4+ T cells in primary biliary cirrhosis.:Dig Dis Sci. 2002, 47(6):1209-15.
73. Pesce G, Ciprandi G, Buscaglia S, Fiorino N, Salmaso C, Riccio A M, Canonica G W, Bagnasco M.:Preliminary evidence for ‘aberrant’ expression of the leukocyte integrin LFA-1 (CD11a/CD18) on conjunctival epithelial cells of patients with mite allergy.:Int Arch Allergy Immunol. 2001, 125(2):160-3.
74. Whitcup S M, Chan C C, Kozhich A T, Magone M T.:Blocking ICAM-1 (CD54) and LFA-1 (CD11a) inhibits experimental allergic conjunctivitis.:Clin Immunol. 1999, 93(2):107-13.
75. Tomita K, Tanigawa T, Yajima H, Sano H, Fukutani K, Hitsuda Y, Matsumoto Y, Sasaki T.:Expression of adhesion molecules on mononuclear cells from individuals with stable atopic asthma.:Clin Exp Allergy. 1997, 27(6):664-71.
76. Asakura K, Saito H, Kataura A.:In vivo effects of monoclonal antibody against ICAM-1 and LFA-1 on antigen-induced nasal symptoms and eosinophilia in sensitized rats.:Int Arch Allergy Immunol. 1996, 111(2):156-60.
77. Rote W E, Dempsey E, Maki S, Vlasuk G P, Moyle M.:The role of CD11/CD18 integrins in the reverse passive Arthus reaction in rat dermal tissue.:J Leukoc Biol. 1996, 59(2):254-61.)
78. Winquist R J, Desai S, Fogal S, Haynes N A, Nabozny G H, Reilly P L, Souza D, Panzenbeck M.:The role of leukocyte function-associated antigen-1 in animal models of inflammation.:Eur J. Pharmacol. 2001, 429(1-3):297-302.
78. Murayama M, Yasuda H, Nishimura Y, Asahi M.:Suppression of mouse contact hypersensitivity after treatment with antibodies to leukocyte function-associated antigen-1 and intracellular adhesion molecules-1.:Arch Dermatol Res. 1997, 289(2):98-103.
79. Hakugawa J, Bae S J, Tanaka Y, Katayama I.:The inhibitory effect of anti-adhesion molecule antibodies on eosinophil infiltration in cutaneous late phase response in Balb/c mice sensitized with ovalbumin (OVA).:J. Dermatol. 1997, 24(2):73-9.
80. Bloemen P G, Buckley T L, van den Tweel M C, Henricks P A, Redegeld F A, Koster A S, Nijkamp F P.:LFA-1, and not Mac-1, is crucial for the development of hyperreactivity in a murine model of nonallergic asthma.:Am J Respir Crit Care Med. 1996, 153(2):521-9.
81. Tanaka Y, Takahashi A, Arai I, Inoue T, Higuchi S, Otomo S, Watanabe K, Habu S, Nishimura T.:Prolonged inhibition of an antigen-specific IgE response in vivo by monoclonal antibody against lymphocyte function-associated antigen-1.:Eur J. Immunol. 1995, 25(6):1555-8.
82. Marubayashi S, Oshiro Y, Maeda T, Fukuma K, Okada K, Hinoi T, Ikeda M, Yamada K, Itoh H, Dohi K.:Protective effect of monoclonal antibodies to adhesion molecules on rat liver ischemia-reperfusion injury.:Surgery. 1997, 122(1):45-52.
83. Tajra L C, Martin X, Margonari J, Blanc-Brunat N, Ishibashi M, Vivier G, Panaye G, Steghens J P, Kawashima H, Miyasaka M, Treille-Ritouet D, Dubernard J M, Revillard J P.:In vivo effects of monoclonal antibodies against rat beta(2) integrins on kidney ischemia-reperfusion injury.:J Surg Res. 1999, 87(1):32-8.
84. Da Silva M, Petruzzo P, Virieux S, Tiollier J, Badet L, Martin X.:A primate model of renal ischemia-reperfusion injury for preclinical evaluation of the antileukocyte function associated antigen 1 monoclonal antibody odulimonab.:J Urol. 2001, 166(5):1915-9.
85. Kelly K J, Williams W W, Colvin R B, Bonventre J V.:Antibody to intercellular adhesion molecule 1 protects the kidney against ischemic injury.:Proc Natl Acad Sci USA. 1994, 91:812-6.
86. Childs E W, Smalley D M, Moncure M, Miller J L, Cheung L Y.:Effect of LFA-1beta antibody on leukocyte adherence in response to hemorrhagic shock in rats.:Shock. 2000, 14(1):49-52.
87. DeMeester S R, Molinari M A, Shiraishi T, Okabayashi K, Manchester J K, Wick M R, Cooper J D, Patterson G A.:Attenuation of rat lung isograft reperfusion injury with a combination of anti-ICAM-1 and anti-beta2 integrin monoclonal antibodies.:Transplantation. 1996, 62(10): 1477-85.
88. Barouch F C, Miyamoto K, Allport J R, Fujita K, Bursell S E, Aiello L P, Luscinskas F W, Adamis A P.:Integrin-mediated neutrophil adhesion and retinal leukostasis in diabetes.:Invest Ophthalmol Vis Sci. 2000, 41(5):1153-8.)
89. Kobashigawa J A, Katznelson S, Laks H, Johnson J A, Yeatman L, Wang X M, Chia D, Terasaki P I, Sabad A, Cogert G A, et al.:Effect of pravastatin on outcomes after cardiac transplantation.:N Engl J. Med. 1995, 333(10):621-7.
90. Dedrick R L, Walicke P, Garovoy M.:Anti-adhesion antibodies efalizumab, a humanized anti-CD11a monoclonal antibody.:Transpl Immunol. 2002, 9(2-4):181-6.
91. Werther W A, Gonzalez T N, O'Connor S J, McCabe S, Chan B, Hotaling T, Champe M, Fox J A, Jardieu P M, Berman P W, Presta L G.:Humanization of an anti-lymphocyte function-associated antigen (LFA)-1 monoclonal antibody and reengineering of the humanized antibody for binding to rhesus LFA-1.:J. Immunol. 1996, 157(11):4986-95.
92. Pietersz G A, Sandrin M S, Ling S, Li Y Q, McKenzie I F C.:LFA-1 and ICAM-1 antibody-idarubicin conjugates separately prolong murine cardiac allograft survival.:Transpl Immunol. 2001, 9(1):7-11.
93. Ozer K, Siemionow M.:Combination of anti-ICAM-1 and anti-LFA-1 monoclonal antibody therapy prolongs allograft survival in rat hind-limb transplants.:J Reconstr Microsurg. 2001, 17(7):511-7; discussion 518.
94. Morikawa M, Brazelton T R, Berry G J, Morris R E.:Prolonged inhibition of obliterative airway disease in murine tracheal allografts by brief treatment with anti-leukocyte function-associated antigen-1 (CD11a) monoclonal antibody.:Transplantation. 2001, 71(11): 1616-21.
95. Bowles M J, Pockley A G, Wood R F.:Effect of anti-LFA-1 monoclonal antibody on rat small bowel allograft survival and circulating leukocyte populations.:Transpl Immunol. 2000, 8(1):75-80.
96. Grochowiecki T, Gotoh M, Dono K, Takeda Y, Sakon M, Yagita H, Okumura K, Miyasaka M, Monden M.:Induction of unresponsiveness to islet xenograft by MMC treatment of graft and blockage of LFA-1/ICAM-1 pathway.:Transplantation. 2000, 69(8): 1567-71.
97. Bashuda H, Takazawa K, Tamatani T, Miyasaka M, Yagita H, Okumura K.:Induction of persistent allograft tolerance in the rat by combined treatment with anti-leukocyte function-associated antigen-1 and anti-intercellular adhesion molecule-1 monoclonal antibodies, donor-specific transfusion, and FK506.:Transplantation. 1996, 62(1):117-22.
98. Guerette B, Skuk D, Celestin F, Huard C, Tardif F, Asselin I, Roy B, Goulet M, Roy R, Entman M, Tremblay J P.:Inhibition by anti-LFA-1 of acute myoblast death following transplantation.:J Immunol. 1997, 159(5):2522-31.
99. Zhang M, Caragine T, Wang H, Cohen P S, Botchkina G, Soda K, Bianchi M, Ulrich P, Cerami A, Sherry B, Tracey K J. Spermine inhibits proinflammatory cytokine synthesis in human mononuclear cells: a counterregulatory mechanism that restrains the immune response.: J Exp Med. 1997 185(10):1759-68.).
100. Feng C G, Britton W J, Palendira U, Groat N L, Briscoe H, Bean A G.: Up-regulation of VCAM-1 and differential expansion of beta integrin-expressing T lymphocytes are associated with immunity to pulmonary Mycobacterium tuberculosis infection.: J. Immunol. 2000 164(9):4853-60.
101. Okayama Y, Kirshenbaum A S, Metcalfe D D.: Expression of a functional high-affinity IgG receptor, Fc gamma RI, on human mast cells: Up-regulation by IFN-gamma.: J. Immunol. 2000 164(8):4332-9.
102. Tang Q, Hendricks R L.: Interferon gamma regulates platelet endothelial cell adhesion molecule 1 expression and neutrophil infiltration into herpes simplex virus-infected mouse corneas.: J Exp Med. 1996 184(4):1435-47.
103. Collins T, Read M A, Neish A S, Whitley M Z, Thanos D, Maniatis T.: Transcriptional regulation of endothelial cell adhesion molecules: NF-kappa B and cytokine-inducible enhancers.: FASEB J. 1995 9(10):899-909.
104. Melder R J, Walker E, Herberman R B, Whiteside T L.:Adhesion characteristics of human interleukin 2-activated natural killer cells.:Cell Immunol. 1991, 132(1):177-92.
105. Bardócz S, Grant G, Brown D S, Ralph A, and Pusztai A.:Polyamines in food—implications for growth and health.:J. Nutr. Biochem. 1993. 4:66-71.
106. Deloyer P, Peulen O, Dandrifosse G.:Dietary polyamines and non-neoplastic growth and disease.:Eur J Gastroenterol Hepatol. 2001, 13(9):1027-32.
107. Sousadias M G, Smith T K.:Toxicity and growth-promoting potential of spermine when fed to chicks.:J Anim Sci. 1995, 73(8):2375-81.
108. Jeevanadam M, Holaday N J, Begay C K, Petersen S R.:Nutritional efficacy of a spermidine supplemented diet.:Nutrition. 1997, 13(9):788-94.)
109. Til H P, Falke H E, Prinsen M K, Willems M I.:Acute and subacute toxicity of tyramine, spermidine, spermine, putrescine and cadaverine in rats.:Food Chem Toxicol. 1997, 35(3-4):337-48

1. (canceled)

2. An LFA-1 inhibitor comprising at least one member selected from the group consisting of polyamines represented by general formula (1) and pharmaceutical acceptable salts thereof:
NH₂—(CH₂)m1-(NH)p1-(CH₂)m2-(NH)p2-(CH₂)m3-(NH)p3-(CH₂)m4-(NH)p4-(CH₂)m5-NH₂ (1)
wherein each of m1 to m5 is independently an integer of 0 to 7, at least two of m1 to m5 exceed 0, the sum of m1+m2+m3+m4+m5 is 2 or more but less than 18, at least one of p1, p2, p3 and p4 is 1, and each of others independently represents 0 or 1.

3. The LFA-1 inhibitor of claim 2, wherein each of m1 to m5 independently represents an integer 0 to 5 and the sum of m1+m2+m3+m4+m5 is 2 or more but less than 17 in formula (1).

4. The LFA-1 inhibitor of claim 3, wherein the sum of m1+m2+m3+m4+m5 is 4 or more but less than 16 in formula (1).

5. The LFA-1 inhibitor of claim 2, wherein m1 is an integer of 2 to 7, m2 is an integer of 2 to 7, m3, m4 and m5 are 0, p 1 is 1, and p2, p3 and p4 are 0 in formula (1).

6. The LFA-1 inhibitor of claim 2, wherein m1 is an integer of 3 to 5, m2 is an integer of 2 to 5, m3, m4 and m5 are 0, p1 is 1, and p2, p3 and p4 are 0 in formula (1).

7. The LFA-1 inhibitor of claim 2, wherein m1 is an integer of 2 to 7, m2 and m3 are an integer of 2 to 7, m4 and m5 are 0, p1 and p2 are 1, and p3 and p4 are 0 in formula (1).

8. The LFA-1 inhibitor of claim 2, wherein m1 is an integer of 3 to 5, m2 and m3 are an integer of 2 to 5, m4 and m5 are 0, p1 and p2 are 1, and p3 and p4 are 0 in formula (1).

9. The LFA-1 inhibitor of claim 2, wherein m1, m2, m3 and m4 are an integer of 2 to 7, m5 is 0, p1, p2 and p3 are 1, and p4 is 0 in formula (1).

10. The LFA-1 inhibitor of claim 2, wherein m1 is an integer of 3 to 5, m2, m3 and m4 are an integer of 2 to 5, m5 is 0, p1, p2 and p3 are 1, and p4 is 0 in formula (1).

11. The LFA-1 inhibitor of claim 2, wherein the polyamine is selected from the group consisting of 3,3′-iminobispropylamine, N-aminobutyl-1,3-diaminopropane, 4,4′-iminobisbutylamine and N-aminopentyl-1,3-diaminopropane.

12. The LFA-1 inhibitor of claim 2, wherein the polyamines is N-aminobutyl-1,3-diaminopropane.

13. The LFA-1 inhibitor of claim 2, wherein the polyamine is selected from the group consisting of 4,8-diazaundecane-1,11-diamine, 4,9-diazadodecane-1,12-diamine, 4,8-diazadodecane-1,12-diamine, 5,9-diazatridecane-1,13-diamine, 4,8-diazatridecane-1,13-diamine, 4,10-diazatridecane-1,13-diamine, 4,9-diazatridecane-1,13-diamine, 5,9-diazatridecane-1,13-diamine and 5,9-diazatetradecane-1,14-diamine.

14. The LFA-1 inhibitor of claim 2, wherein the polyamine is selected from the group consisting of 4,9-diazadodecane-1,12-diamine and 4,8-diazadodecane-1,12-diamine.

15. The LFA-1 inhibitor of claim 2, wherein the polyamine is 4,9-diazadodecane-1,12-diamine.

16. The LFA-1 inhibitor of claim 2, wherein the polyamine is selected from the group consisting of 4,8,12-triazapentadecane-1,15-diamine, 4,8,12-triazahexadecane-1,16-diamine, 4,9,13-triazaheptadecane-1,17-diamine, 4,9,14-triazaoctadecane-1,18-diamine, 5,9,13-triazaheptadecane-1,17-diamine, 5,9,14-triazaoctadecane-1,18-diamine, 4,9,14-triazaoctadecane-1,18-diamine, and 5,10,14-triazaoctadecane-1,18-diamine.

17. The LFA-1 inhibitor of claim 2, wherein the polyamine is 4,8,12-triazapentadecane-1,15-diamine or 4,8,12-triazahexadecane-1,16-diamine.

18. The LFA-1 inhibitor of claim 2, wherein the polyamine is selected from the group consisting of 4,8,12,16-tetraazanonadecane-1,19-diamine, 4,8,12,16-tetraazaicosane-1,20-diamine, 4,8,12,17-tetraazaicosane-1,20-diamine and 4,8,13,17-tetraazaicosane-1,20-diamine.

19. The LFA-1 inhibitor of claim 2, wherein the polyamine is 4,8,12,16-tetraazanonadecane-1,19-diamine, or 4,8,12,16-tetraazaicosane-1,20-diamine.

20. A pharmaceutical composition for the treatment of arteriosclerosis comprising the LFA-1 inhibitor of claim 2.

21. A pharmaceutical composition for the inhibition of rejection comprising the LFA-1 inhibitor of claim 2.

22. A pharmaceutical composition for the treatment of autoimmune disease comprising the LFA-1 inhibitor of claim 2.

23. A pharmaceutical composition for the treatment of allergy comprising the LFA-1 inhibitor of claim 2.

24. A pharmaceutical composition for the treatment of ischemic reperfusion injury comprising the LFA-1 inhibitor of claim 2.

25. A pharmaceutical composition for the treatment of diabetic retinopathy comprising the LFA-1 inhibitor of claim 2.

26. A method for the treatment of diseases selected from the group consisting of arteriosclerosis, autoimmune disease, allergy, ischemic reperfusion injury, and diabetic retinopathy comprising administrating the LFA-1 inhibitor of claim 2.

27. A method for the inhibition of diseases selected from the group consisting of arteriosclerosis, autoimmune disease, allergy, ischemic reperfusion injury, and diabetic retinopathy comprising administrating the LFA-1 inhibitor of claim 2.

28. The method according to claim 26, wherein the LFA-1 inhibitor is administrated in the range of 0.01-100 mg/day per 1 Kg of body weight of a patient.

29. The method of claim 26, wherein the LFA-1 inhibitor is administrated in the range of 0.05-40 mg/day per 1 Kg of body weight of a patient.

30. The method of claim 26, wherein the LFA-1 inhibitor is administrated in the range of 0.05-4 mg/day per 1 Kg of body weight of a patient.

31. A method for the inhibition of rejection comprising administrating the LFA-1 inhibitor of claim 2.

32. The method of claim 31, wherein the LFA-1 inhibitor is administrated in the range of 0.01-100 mg/day per 1 Kg of body weight of a patient.

33. The method of claim 31, wherein the LFA-1 inhibitor is administrated in the range of 0.01-40 mg/day per 1 Kg of body weight of a patient.

34. The method of claim 31, wherein the LFA-1 inhibitor is administrated in the range of 0.05-4 mg/day per 1 Kg of body weight of a patient.

35. A method for the inhibition of rejection of transplant organ, wherein the method comprises perfusing or preserving organs with a perfusate or a preservative solution containing 1 μM to 10 mM of the polyamines of claim 2 when the transplant organ is perfused or preserved.
